(19) United States
(12) Patent Application Publication
(10) Pub. No.: US 2022/0105202 A1
(43) Pub. Date: Apr. 7, 2022

(54) UNIVERSAL MULTI-FUNCTIONAL GSH-RESPONSIVE SILICA NANOPARTICLES FOR DELIVERY OF BIOMOLECULES INTO CELLS

(71) Applicant: Wisconsin Alumni Research Foundation, Madison, WI (US)

(72) Inventors: Shaoqin GONG, Middleton, WI (US); Yuyuan Wang, Madison, WI (US)

(21) Appl. No.: 17/501,635

(22) Filed: Oct. 14, 2021

Related U.S. Application Data
(63) Continuation-in-part of application No. PCT/US2021/032949, filed on May 18, 2021.
(60) Provisional application No. 63/026,484, filed on May 18, 2020.

(51) Int. Cl. A61K 48/00; A61K 9/51; A61K 47/69; A61P 25/00; C08G 83/00

(52) U.S. Cl. CPC A61K 48/0041; A61K 9/5146; C08G 83/001; A61P 25/00; A61K 47/6935

(57) ABSTRACT

The present technology provides a nanoparticle comprising: the polysiloxanes comprise silyloxy subunits having the structure (I) as shown herein, wherein R^{a} at each occurrence is independently selected from a bond to a Si of another polysiloxane chain or a C₁₋₁₂ alkyl group; Rⁱ at each occurrence is independently selected from the group consisting of C₁₋₁₂ alkyl and C₂₋₁₂ alkenyl groups, optionally substituted with a substituent selected from the group consisting of halogen and NR¹₂, wherein each occurrence of R¹ is independently selected from H or a C₁₋₁₂ alkyl group, or two R¹ groups, together with the N atom to which they are attached, form a pyrrolidine or piperidine ring; the crosslinks between polysiloxanes comprise disulfide linkages, the nanoparticle comprises an exterior surface comprising surface-modifying groups attached to and surrounding the silica network, wherein the surface-modifying groups comprise polyethylene glycol (PEG), polysarcosine, polyzwitterion, polycation, polyanion, or combinations of two or more thereof; and the nanoparticle has an average diameter of 15 nm to 200 nm. The nanoparticles herein may include biomolecules such as polynucleic acids, proteins, and complexes thereof, e.g., Cas9 RNP.

### CROSS REFERENCE TO RELATED APPLICATION

This continuation-in-part application claims the benefit of and priority to PCT Application No. PCT/US2021/032949, filed May 18, 2021, which in turn claims priority to U.S. Provisional Patent Application No. 63/026,484, filed on May 18, 2020, the entire contents of each of which are incorporated herein by reference in their entireties.

### FIELD

The present technology relates generally to the field of nanoplatform delivery systems. The delivery systems include a multi-functional GSH-responsive silica nanoparticles (SNPs) suitable for the delivery of biomolecules to cells. The nanoparticles include disulfide crosslinks and other functionality that permit them to efficiently deliver hydrophilic charged polynucleic acids, polypeptides (including proteins) and complexes of polypeptides and nucleic acids such s RNP to cells. Methods of preparing and using the nanoparticles are also provided.

### STATEMENT OF GOVERNMENT SUPPORT

This invention was made with government support under 1844701 awarded by the National Science Foundation. The government has certain rights in the invention.

### BACKGROUND

Safe and efficient delivery of biomacromolecules (e.g., nucleic acids and CRISPR ribonucleoproteins (RNPs)) to target cells for therapeutic purposes remains a challenge. Nucleic acids, including DNA and mRNA, are widely used for gene therapy because of their relatively rapid and safe protein production. CRISPR-Cas9 RNPs can achieve genome editing by introducing gene deletion, correction, and/or insertion with high efficiency and specificity. However, under physiological conditions, naked nucleic acids and RNPs are prone to enzymatic degradation. Moreover, the transfection/gene editing efficiency is negligible due to the lack of cellular uptake and endosomal escape capability. In addition, efficient delivery of protein/nucleic acid complexes such as RNP or RNP together with single-stranded oligonucleotide DNA (i.e., RNP+ssODN) for genome editing is hindered by its heterogenous charges and complicated structures. To address such issues, non-viral nanovectors have been investigated for the delivery of biomacromolecules. Nonetheless, current state-of-the-art non-viral nanovectors often suffer from low payload encapsulation content/efficiency, high cytotoxicity and insufficient in vivo stability.

### SUMMARY OF THE INVENTION

As disclosed herein, the present technology provides new multi-functional GSH-responsive SNPs that safely and efficiently deliver biomolecules into cells, particularly animal cells. In various aspects and embodiments the present SNP technology provides one or more: (1) high loading content and loading efficiency, while maintaining the payload activity, (2) small NP size (e.g., hydrodynamic diameter<500 nm and even <200 nm or <100 nm), (3) versatile surface chemistry (e.g., ligand conjugation) to facilitate the payload delivery to target cells, (4) excellent biocompatibility, (5) efficient endo/lysosomal escape capability, (6) rapid payload release in the target cells, and (7) ease of handling, storage, and transport.

Thus, in one aspect, the present technology provides a nanoparticle comprising: a silica network comprising crosslinked polysiloxanes, wherein the crosslinks between polysiloxanes comprise disulfide linkages, the polysiloxanes optionally bear weakly basic functional groups, the nanoparticle comprises an exterior surface comprising surface-modifying groups attached to and surrounding the silica network, wherein the surface-modifying groups comprise polyethylene glycol (PEG), polysarcosine, polyzwitterion or combinations of two or more thereof; and the nanoparticle has an average diameter of 15 nm to 500 nm.

In another aspect, the present technology provides a nanoparticle comprising: a silica network comprising crosslinked polysiloxanes, wherein the crosslinks between polysiloxanes comprise disulfide linkages, the polysiloxanes optionally bear weakly basic functional groups, the nanoparticle comprises an exterior surface comprising surface-modifying groups attached to and surrounding the silica network, wherein the surface-modifying groups comprise polyethylene glycol (PEG), polysarcosine, polycation, polyanion, polyzwitterion or combinations of two or more of thereof; the surface potential of the nanoparticle ranges from −45 mV to +45 mV; and the nanoparticle has an average diameter of 15 nm to 500 nm.

In another aspect, the present technology provides a nanoparticle comprising: a silica network comprising crosslinked polysiloxanes, wherein the polysiloxanes comprise silyloxy subunits having the structure (I)

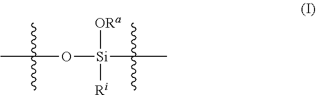

wherein
R^{a} at each occurrence is independently selected from a bond to a Si of another polysiloxane chain or a C₁₋₁₂ alkyl group;
Rⁱ at each occurrence is independently selected from the group consisting of C₁₋₁₂ alkyl and C₂₋₁₂ alkenyl groups, optionally substituted with a substituent selected from the group consisting of halogen and NR¹₂, wherein each occurrence of R¹ is independently selected from H or a C₁₋₁₂ alkyl group, or two R¹ groups, together with the N atom to which they are attached, form a pyrrolidine or piperidine ring;
the crosslinks between polysiloxanes comprise disulfide linkages,
the nanoparticle comprises an exterior surface comprising surface-modifying groups attached to and surrounding the silica network, wherein the surface-modifying groups comprise polyethylene glycol (PEG), polysarcosine, polyzwitterion, polycation, polyanion, or combinations of two or more thereof; and
the nanoparticle has an average diameter of 15 nm to 200 nm.

In another aspect, the present technology provides SNPs comprising a water-soluble biomolecule (a payload), such as polynucleic acids, proteins, and complexes thereof such as Cas9 RNP. In yet another aspect, the present technology provides a method of delivering a water-soluble biomolecule into a cell comprising exposing the cell to a nanoparticle of any aspect or embodiment as disclosed herein. In still another aspect, the present technology provides a method of treating a condition or disorder in a subject that may be ameliorated by a biomolecule comprising administering to the subject an effective amount of a nanoparticle including the biomolecule of any aspect or embodiment disclosed herein. In any embodiments, the condition or disorder occurs in, but not limited to the central nervous system of the subject, and the nanoparticle comprises glucose and/or RVG targeting ligands.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIGS. 1A-1F schematically illustrate the synthesis and mechanism of action of an illustrative embodiment of the present technology. FIG. 1A schematically illustrates a non-limiting embodiment of the present SNPs (4-arm) for the delivery of various water-soluble biomolecules such as polynucleic acids (e.g., DNA and mRNA) and CRISPR-Cas9 genome editing machinery (e.g., RNP, RNP+ssODN). FIG. 1B schematically illustrates the synthesis of one embodiment of SNPs via a water-in-oil emulsion method, including synthesis of silica network, PEGylation and ATRA-conjugation of SNPs. FIG. 1C is a schematic illustration of the intracellular trafficking pathways of a nonlimiting embodiment of SNPs of the present technology. FIG. 1D shows ¹H NMR (CDCl₃) spectrum of 3,5-O-benzylidene-1,2-O-isopropylidene-α D-glucofuranoside (BIG). FIG. 1E shows ¹H NMR (CDCl₃) spectrum of (3-arm) silane-PEG-Glu. FIG. 1F shows ¹H NMR (CDCl₃) spectrum of (3-arm) silane-PEG-RVG.

FIGS. 2A-2F shows 4-arm SNP characterization data for an illustrative embodiment of the present technology. FIG. 2A shows size distribution of an SNP of Example 3 measured by DLS. FIG. 2B is a transmission electron microscopy micrograph of DNA-loaded SNPs of Example 3. FIG. 2C shows graphs charting the effect of (1) molar ratio of TESPIC, and (2) surface charge in DNA-delivery by SNPs (Example 4). The transfection efficiencies of the various formulations were evaluated by quantification of RFP-positive HEK293 cells 48 h post treatment. NS: not significant; *: p<0.05; **: p<0.01; n=3. FIG. 2D shows graphs charting the effect of (1) molar ratio of TESPIC, and (2) surface charge on mRNA delivery by SNPs (Example 4). The transfection efficiencies of the various formulations were evaluated by quantification of RFP-positive HEK293T cells 48 h after treatments. NS: not significant; ****: p<0.0001; n=3. FIG. 2E is a graph showing the effects of GSH concentration in a cell culture medium on the DNA transfection efficiency of SNP-PEG. FIG. 2F is a graph showing the mRNA delivery efficiency of SNP-PEG after storage at different conditions. NS: not significant; *: p<0.05; **: p<0.01;****: p<0.0001; n=3.

FIG. 3 shows confocal laser scanning micrographs demonstrating colocalization of ATTO-550-tagged RNP and endo/lysosomes at 0.5 h, 2 h, and 6 h post-treatment times in HEK 293 cells.

FIGS. 4A-4F show the delivery efficiency of nucleic acids and CRISPR-Cas9 genome-editing machineries by illustrative embodiments of 4-arm SNPs of the present technology. FIGS. 4A and 4B show, respectively, the transfection efficiency of the DNA- and mRNA-loaded SNP-PEG in HEK293 cells. FIG. 4C shows the gene deletion efficiency of RNP-loaded SNP-PEG in GFP-expressing HEK 293 cells. FIG. 4D schematically illustrates HDR at a BFP reporter locus induced by the RNP+ssODN. Sequences of unedited (BFP) and edited (GFP) loci are shown. The protospacer adjacent motif sequence of RNP is underlined and the RNP cleavage site is marked by an arrow. FIG. 4E shows the gene-correction efficiency of RNP+ssODN co-encapsulated SNP-PEG in BFP-expressing HEK 293 cells. NS: not significant *: p<0.05; **: p<0.01; n=3. FIG. 4F is a graph showing the viability of HEK 293 cells treated with DNA-loaded SNP-PEG with different concentrations and DNA-complexed Lipo 2000. NS: not significant; ****: p<0.0001; n=7.

FIGS. 5A-5E show the nucleic acid and RNP delivery efficiency of 4-arm SNPs in Ai14 mice via subretinal injection (Example 7). FIG. 5A shows the tdTomato locus in the Ai14 reporter mouse. TdTomato expression can be achieved by Cre-Lox recombination. FIG. 5B schematically illustrates subretinal injection targeting the RPE tissue. FIG. 5C shows the stop cassette containing 3 Ai14 sgRNA target sites prevents downstream tdTomato expression. Excision of 2 SV40 polyA blocks by Ai14 RNP results in tdTomato expression. FIG. 5D shows the efficient delivery of Cre-mRNA by SNP-PEG-ATRA in mouse RPE. D1, RPE floret of eyes subretinally injected with Cre-mRNA-encapsulated SNPs; D2, 20× magnification images of tdTomato+ RPE tissue; D3, RPE floret of PBS controls. FIG. 5E shows the efficient delivery of RNP by SNP-PEG-ATRA in mouse RPE. E1, RPE floret of mouse eyes subretinally injected with Ai14 RNP-encapsulated SNPs; E2, 20× magnification images of tdTomato+ RPE tissue; E3, RPE floret of Ai14 mice injected with negative control SNP-PEG-ATRA (SNP-PEG-ATRA encapsulating RNP with negative control sgRNA). The whole RPE layer was outlined with a white dotted line.

FIG. 6 is photomicrographs showing the internalization of 4-arm SNP-PEG-TAT by hiPSC-RPE cells according to illustrative embodiments of SNPs of the present technology. FIG. 6 (left to right) shows untreated hiPSC-RPE cells (i.e., control) at 20× and 50× (lower panel) and RNP+ssODN-loaded SNP-PEG-TAT uptake by iPSC-RPE after 4 days of treatment with RNP dosages of 3 μg, 6 μg, and 12 μg per well, in a superimposed image (i.e., bright field+ATTO-488) on the upper panel and the reconstituted z-stack fluorescence image on the lower panel.

FIGS. 7A-7B show in vivo 4-arm SNP delivery of nucleic acid and RNP by systemic administration according to illustrative embodiments of SNPs of the present technology. FIGS. 7A and 7B show, respectively, tissue homogenization of Ai14 mice injected with Cre-mRNA or RNP encapsulated SNP-PEG or SNP-PEG-GalNAc detected and analyzed ex vivo by tdTomato fluorescence.

FIG. 8 shows the blood biochemical profile of SNP-PEG and SNP-PEG-GalNAc injected mice according to illustrative embodiments of SNPs of the present technology. NS: not significant; n=3.

FIGS. 9A-9D show in vitro delivery efficiency of various 3-arm and 4-arm SNPs. (9A) and (9B) Transfection efficiency of the (9A) DNA- and (9B) mRNA-loaded SNPs in HEK293 cells. (9C) Gene editing efficiency of RNP-loaded SNPs in GFP-expressing HEK 293 cells. (9D) Viability of HEK 293 cells treated with DNA-loaded SNPs and DNA-complexed Lipo 2000. For viability study, statistical difference was calculated between each group and untransfected cells (UT). Data are presented as mean±SD. Statistical significance (ρ value) was calculated via one-way ANOVA with a Tukey post hoc test.

FIGS. 10A-10B show a schematic illustrations of: (10A) SNPs formed by different silica reagents. R=nonhydrolyzable inactive arm. (10B) Schematic illustration of systemic delivery of SNPs into the brain.

FIGS. 11A-11E show results of an in vivo study of the genome editing efficiency of RNP-SNP1 conjugated with different types/amounts of targeting ligands (glucose and/or RVG) after systemic administration. (11A-11B) Optimization of the surface targeting ligands using RNP-SNP1. TdTomato signal in (11A) brain and (11B) major organs were analyzed by ex vivo IVIS imaging. (11C-11E) FACS analysis of edited cell types in the brain. TdTomato+ cells co-localizing with (11C) neurons, (11D) Astrocytes, and (11E) BCECs. Data are presented as mean±SD. Statistical significance (p value) was calculated via one-way ANOVA with a Tukey post hoc test.

FIGS. 12A-12D show Cre mRNA-encapsulated SNPs induced tdTomato expression in Ai14 mouse brain. (12A-12B) SNPs with different formulations showed high brain accumulation and Cre mRNA delivery to the brain. (12A) Ex vivo IVIS imaging of brains. (12B) Ex vivo IVIS imaging of major organs. (12C-12D) Representative CLSM images of the brains of Ai14 mice intravenously injected with PBS, or Cre mRNA-encapsulated SNPs (i.e., mRNA-SNP1-Glu+RVG, mRNA-SNP2-Glu+RVG, mRNA-SNP6-Glu+RVG and mRNA-SNP7-Glu+RVG) at +1.18 mm Bregma (12C) and −1.70 mm Bregma (12D). Blue, DAPI staining nuclei; red, tdTomato.

FIGS. 13A-13F show Cre mRNA-encapsulated SNPs induced tdTomato expression in neurons and other cell types. (13A-13C) Representative CLSM images of (13A) cortex, (13B) striatum, and (13C) hippocampus with tdTomato positive cells co-localizing with neuron and BCEC markers. Scale bar: 50 μm. (13D-13F) FACS analysis of tdTomato positive neurons (13D), astrocytes (13E), and BCECs (13F). Data are presented as mean±SD. Statistical significance (p value) was calculated via one-way ANOVA with a Tukey post hoc test.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; sulfates; phosphates; amines; N-oxides; hydrazines; hydrazides; hydrazones; azides (—N₃); amides; ureas; amidines; guanidines; enamines; imides; imines; nitro groups (—NO₂); nitriles (—CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, amidinealkyl, guanidinealkyl, alkoxyalkyl, carboxyalkyl, and the like.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═H(CH₃), —CH═(CH₃)₂, —C(CH₃)CH₂, —C(CH₃═CH(CH₃), —C(CH₂CH₃CH₂, among others. Representative substituted alkenyl groups may be mono-substituted or substituted more than once, such as, but not limited to, mono-, di- or tri-substituted with substituents such as those listed above for alkyl.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic carbon-containing ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, oxadiazolonyl (including 1,2,4-oxazol-5(4H)-one-3-yl), isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic carbon-containing ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent alkenyl groups are alkenylene groups, and so forth. Substituted groups having a single point of attachment to a compound or polymer of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, cycloalkyl, aryl, aralkyl, heterocyclylalkyl or heterocyclyl group as defined herein. Amido groups therefore include but are not limited to carbamoyl groups (—C(O)NH₂) (also referred to as “carboxamido groups”) and formamido groups (—NHC(O)H). In some embodiments, the amide is —NR⁷¹C(O)—(C₁₋₅ alkyl) and the group is termed “alkanoylamino.”

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, aryl aralkyl, heterocyclyl or heterocyclylalkyl group as defined herein. It will be understood that amidines may exist in protonated forms in certain aqueous solutions or mixtures and are examples of charged functional groups herein.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, cycloalkyl, aryl, aralkyl, heterocyclylalkyl or heterocyclyl group as defined herein. In some embodiments, the amine is NH₂, alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. It will be understood that amines may exist in protonated forms in certain aqueous solutions or mixtures and are examples of charged functional groups herein.

The term “carboxyl” or “carboxylate” as used herein refers to a —COOH group or its ionized salt form. As such, it will be understood that carboxyl groups are examples of charged functional groups herein.

The term “ester” as used herein refers to —COOR⁷⁰ and —C(O)O-G groups. R⁷⁰ is a substituted or unsubstituted alkyl, cycloalkyl, alkenyl, aryl, aralkyl, heterocyclylalkyl or heterocyclyl group as defined herein. G is a carboxylate protecting group. As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3^{rd} Edition, 1999). Which can be added or removed using the procedures set forth therein and which is hereby incorporated by reference in its entirety and for any and all purposes as if fully set forth herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, aryl aralkyl, heterocyclyl or heterocyclylalkyl group as defined herein. It will be understood that guanidines may exist in protonated forms in certain aqueous solutions or mixtures and are examples of charged functional groups herein.

The term “hydroxyl” as used herein can refer to —OH or its ionized form, —O⁻. A “hydroxyalkyl” group is a hydroxyl-substituted alkyl group, such as HO—CH₂—.

The term “imidazolyl” as used herein refers to an imidazole group or the salt thereof. An imidazolyl may be protonated in certain aqueous solutions or mixtures, and is then termed an “imidazolate.”

The term “phosphate” as used herein refers to —OPO₃H₂ or any of its ionized salt forms, —OPO₃HR⁸⁴ or —OPO₃R⁸⁴R⁸⁵ wherein R⁸⁴ and R⁸⁵ are independently a positive counterion, e.g., Na⁺, K⁺, ammonium, etc. As such, it will be understood that phosphates are examples of charged functional groups herein.

The term “pyridinyl” refers to a pyridine group or a salt thereof. A pyridinyl may be protonated in certain aqueous solutions or mixtures, and is then termed a “pyridinium group”.

The term “sulfate” as used herein refers to —OSO₃H or its ionized salt form, —OSO₃R⁸⁶ wherein R⁸⁶ is a positive counterion, e.g., Na⁺, K⁺, ammonium, etc. As such, it will be understood that sulfates are examples of charged functional groups herein.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, and R⁸² are each independently a substituted or unsubstituted alkyl, cycloalkyl, alkenyl, aryl aralkyl, heterocyclyl or heterocyclylalkyl group as defined herein. In some embodiments the sulfide is an alkylthio group, —S-alkyl. R⁸³ includes H or, when the sulfonyl is ionized (i.e., as a sulfonate), a positive counterion, e.g., Na⁺, K⁺, ammonium or the like. As such, it will be understood that sulfonyls are examples of charged functional groups herein.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

As used herein, “ribonucleoprotein” or “RNP” refers to a complex between an RNA-binding protein and RNA in which the RNA binds specifically (as opposed to non-specific binding) to the protein. Examples of ribonucleoproteins include CRISPR-associated proteins, e.g., Cas9, Cas12, Cas13, Cas14 and Case.

As used herein, “Cas9” and “Cas9 polypeptide” refer to the complex of Cas9 proteins, and variants thereof having nuclease activity, with RNA (i.e., sgRNA, or crRNA and tracrRNA). Likewise, “Cas12” refers to the complex of Cas12 proteins and variants thereof having nuclease activity, with crRNA. “Cas13” refers to the complex of Cas13 proteins and variants thereof having nuclease activity, with RNA (i.e., crRNA). Cas9, Cas12, and Cas13 also include complexes of fusion proteins containing such Cas9, Cas12, and Cas13 proteins and variants thereof. The fused proteins may include those that modify the epigenome or control transcriptional activity. The variants may include deletions or additions, such as, e.g., addition of one, two, or more nuclear localization sequences (such as from SV40 and others known in the art), e.g., 1, 2, 3, 4, 5, 6, 7, 8, 9, 10 such sequences or a range between and including any two of the foregoing values. In any embodiments the Cas9 polypeptide is a Cas9 protein found in a type II CRISPR-associated system. Suitable Cas9 polypeptides that may be used in the present technology include, but are not limited to Cas9 protein from Streptococcus pyogenes (SpCas9), F. novicida (FnCas9), S. aureus (SaCas9), S. thermophiles (StlCas9), N. meningitidis (NmeCas9), and variants thereof. In any embodiments, the Cas9 polypeptide is a wild-type Cas9, a nickase, or comprises a nuclease inactivated (dCas9) protein. In any embodiments the Cas12 polypeptide is a Cas12 protein found in a type V CRISPR-associated system. Suitable Cas12 polypeptides that may be used in the present technology include, but are not limited to Cas12 protein from Lachnospiraceae bacterium MA2020 (LbCas12a), Acidaminococcus sp. BV3L6 (AsCas12a), Bacillus hisaishi (BhCas12b), and variants thereof. In any embodiments, the Cas12 polypeptide is a wild-type Cas12, a nickase, or comprises a nuclease inactivated (dCas12) protein. In any embodiments the Cas13 polypeptide is a Cas13 protein found in a type VI CRISPR-associated system. Suitable Cas13 polypeptides that may be used in the present technology include, but are not limited to Cas13 protein from Leptotrichia wadei (LwaCas13a), Prevotella sp. P5-125 (PspCas13b), Ruminococcus flavefaciens (RfxCas13d), and variants thereof. In any embodiments, the Cas13 polypeptide is a wild-type Cas13, a nickase, or comprises a nuclease inactivated (dCas13) protein. In any embodiments, the Cas9 polypeptide is a fusion protein comprising dCas9. In any embodiments, the Cas12 polypeptide is a fusion protein comprising dCas12. In any embodiments, the Cas13 polypeptide is a fusion protein comprising dCas13. In any embodiments, the fusion protein comprises a transcriptional activator (e.g., VP64), a transcriptional repressor (e.g., KRAB, SID) a nuclease domain (e.g., FokI), base editor (e.g., adenine base editors, ABE), a recombinase domain (e.g., Hin, Gin, or Tn3), a deaminase (e.g., a cytidine deaminase or an adenosine deaminase) or an epigenetic modifier domain (e.g., TET1, p300). In any embodiments, the Cas9, Cas12, or Cas13 includes variants with at least 85% sequence identity, at least 90% sequence identity, at least 95% sequence identity, or even 96%, 97%, 98%, or 99% sequence identity to the wild type Cas9, Cas12, or Cas13, respectively. Accordingly, a wide variety of Cas9, Cas12, and Cas13 proteins may be used as formation of the present NPs is not sequence dependent so long as the Cas9 protein or Cas12 protein can complex with nucleic acids and the resulting RNP has sufficient charged residuals to allow complexation with the amphiphilic polymers of the present technology. Other suitable Cas9 proteins may be found in Karvelis, G. et al. “Harnessing the natural diversity and in vitro evolution of Cas9 to expand the genome editing toolbox,” Current Opinion in Microbiology 37: 88-94 (2017); Komor, A. C. et al. “CRISPR-Based Technologies for the Manipulation of Eukaryotic Genomes,” Cell 168:20-36 (2017); and Murovec, J. et al. “New variants of CRISPR RNA-guided genome editing enzymes,” Plant Biotechnol. J. 15:917-26 (2017), each of which is incorporated by reference herein in their entirety. Other suitable Cas12 proteins may be found in Makarova, Kira S., et al. “Evolutionary classification of CRISPR-Cas systems: a burst of class 2 and derived variants.” Nature Reviews Microbiology 18.2 (2020): 67-83; Strecker, Jonathan, et al. “Engineering of CRISPR-Cas12b for human genome editing.” Nature Comm. 10.1 (2019): 1-8; and Yan, Winston X., et al. “Functionally diverse type V CRISPR-Cas systems.” Science 363.6422 (2019): 88-91, each of which is incorporated by reference herein in their entirety. Other suitable Cas13 proteins may be found in O'Connell, Mitchell R. “Molecular mechanisms of RNA targeting by Cas13-containing type VI CRISPR-Cas systems.” J. Mol. Biol. 431.1 (2019): 66-87, each of which is incorporated by reference herein in their entirety.

“Molecular weight” as used herein with respect to polymers refers to number-average molecular weights (Me) and can be determined by techniques well known in the art including gel permeation chromatography (GPC). GPC analysis can be performed, for example, on a D6000M column calibrated with poly(methyl methacrylate) (PMMA) using triple detectors including a refractive index (RI) detector, a viscometer detector, and a light scattering detector, and N,N′-dimethylformamide (DMF) as the eluent. “Molecular weight” in reference to small molecules and not polymers is actual molecular weight, not number-average molecular weight.

“Organosilica network” refers to a network containing crosslinked polysiloxane polymers. Polysiloxanes of the present technology comprise repeating silicon-containing substructures of which a fraction (e.g., about 0.01 mol % to about 90 mol %, such as 0.1, 1, 5, 10, 20, 30, 40, 50, 60, 70, 80, or 90 mol %, or a range between and including any two of the foregoing values, including about 0.1 mol % to about 90 mol %, about 1 mol % to about 80 mol %, or about 10 mol % to about 90 mol %) of the repeating silicon-containing substructures include one or more crosslinks to another polysiloxane chain. The crosslinks may include disulfide linkages (—S—S—) and siloxane linkages (e.g., —Si—O—Si—). The organosilica network may include silicon atoms with two polymeric attachment points (i.e., the silicon forms part of a linear polysiloxane chain) and/or three and/or four polymeric attachment points (i.e., crosslinks to polysiloxane chains)

A “polysiloxane” as used herein refers to a linear or branched polymer comprising repeating silyloxy subunits attached to each other through siloxane linkages (Si—O—Si). Polysiloxanes may be homopolymers or copolymers, including random copolymers of more than one type of siloxy subunit.

A “cell penetrating peptide” (CPP), also referred to as a “protein transduction domain” (PTD), a “membrane translocating sequence,” and a “Trojan peptide”, refers to a short peptide (e.g., from 3 to about 40 amino acids) that has the ability to translocate across a cellular membrane to gain access to the interior of a cell and to carry into the cells a variety of covalently and noncovalently conjugated cargoes, including the present nanoparticles and the water-soluble biomolecules. CPPs are typically highly cationic and rich in arginine and lysine amino acids. Examples of such peptides include TAT cell penetrating peptide (GRKKRRQRRRPQ); MAP (KLALKLALKALKAALKLA); Penetratin or Antenapedia PTD (RQIKWFQNRRMKWKK); Penetratin-Arg: (RQIRIWFQNRRMRWRR); antitrypsin (358-374): (C SIPPEVKFNKPFVYLI); Temporin L: (FVQWFSKFLGRIL-NH₂); Maurocalcine: GDC(acm) (LPHLKLC); pVEC (Cadherin-5): (LLIILRRRIRKQAHAHSK); Calcitonin: (LGTYTQDFNKFHTFPQTAIGVGAP); Neurturin: (GAAEAAARVYDLGLRRLRQRRRLRRERVRA); Penetratin: (RQIKIWFQNRRMKWKKGG); TAT-HA2 Fusion Peptide: (RRRQRRKKRGGDIMGEWGNEIFGAIAGFLG); TAT (47-57) Y(GRKKRRQRRR); SynB1 (RGGRLSYSRRRFSTSTGR); SynB3 (RRLSYSRRRF); PTD-4 (PIRRRKKLRRL); PTD-5 (RRQRRTSKLMKR); FHV Coat-(35-49) (RRRRNRTRRNRRRVR); BMV Gag-(7-25) (KMTRAQRRAAARRNRWTAR); HTLV-II Rex-(4-16) (TRRQRTRRARRNR); HIV-1 Tat (48-60) or D-Tat (GRKKRRQRRRPPQ); R9-Tat (GRRRRRRRRRPPQ); Transportan (GWTLNSAGYLLGKINLKALAALAKKIL chimera); SBP or Human P1 (MGLGLHLLVLAAALQGAWSQPKKKRKV); FBP (GALFLGWLGAAGSTMGAWSQPKKKRKV); MPG (ac-GALFLGFLGAAGSTMGAWSQPKKKRKV-cya (wherein cya is cysteamine)); MPG(ΔNLS) (ac-GALFLGFLGAAGSTMGAWSQPKSKRKV-cya); Pep-1 or Pep-1-Cysteamine (ac-KETWWETWWTEWSQPKKKRKV-cya); Pep-2 (ac-KETWFETWFTEWSQPKKKRKV-cya); Periodic sequences, Polyarginines (R×N (4<N<17) chimera); Polylysines (K×N (4<N<17) chimera); (Raca)6R; (Rabu)6R; (RG)6R; (RM)6R; (RT)6R; (RS)6R; R10; (RA)6R; and R7.

A “dye” refers to small organic molecules having a molecular weight (actual, not number average) of 2,000 Da or less or a protein which is able to emit light. Non-limiting examples of dyes include fluorophores, chemiluminescent or phosphorescent entities. For example, dyes useful in the present technology include but are not limited to cyanine dyes (e.g., Cy2, Cy3, Cy5, Cy5.5, Cy7, and sulfonated versions thereof), fluorescein isothiocyanate (FITC), ALEXA FLUOR® dyes (e.g., ALEXA FLUOR® 488, 546, or 633), DYLIGHT® dyes (e.g., DYLIGHT® 350, 405, 488, 550, 594, 633, 650, 680, 755, or 800) or fluorescent proteins such as GFP (Green Fluorescent Protein).

The phrase “targeting ligand” refers to a ligand that binds to “a targeted receptor” that distinguishes the cell being targeted from other cells. The ligands may be capable of binding due to expression or preferential expression of a receptor for the ligand, accessible for ligand binding, on the target cells. Examples of such ligands include GE11 peptide, anti-EGFR nanobody, cRGD ((cyclo (RGDfC)), KE108 peptide, octreotide, all-trams-retinoic acid (ATRA), RVG peptide (YTIWMPENPRPGTPCDIFTNSRGKRASNG), glucose, mannitol, folic acid, prostate-specific membrane antigen (PSMA) aptamer, TRC105, a human/murine chimeric IgG1 monoclonal antibody, mannose, cholera toxin B (CTB), hyaluronic acid, and N-acetylgalactosamine (GalNAc). Additional examples of such ligands include Rituximab, Trastuzumab, Bevacizumab, Alemtuzumab, Panitumumab, RGD, DARPins, RNA aptamers, DNA aptamers, analogs of folic acid and other folate receptor-binding molecules, lectins, other vitamins, amino acids, peptide ligands identified from library screens, tumor-specific peptides, tumor-specific aptamers, tumor-specific carbohydrates, tumor-specific monoclonal or polyclonal antibodies, Fab or scFv (i.e., a single chain variable region) fragments of antibodies such as, for example, an Fab fragment of an antibody directed to EphA2 or other proteins specifically expressed or uniquely accessible on metastatic cancer cells, small organic molecules derived from combinatorial libraries, growth factors, such as EGF, FGF, insulin, and insulin-like growth factors, and homologous polypeptides, somatostatin and its analogs, transferrin, lipoprotein complexes, bile salts, selecting, steroid hormones, Arg-Gly-Asp containing peptides, microtubule-associated sequence (MTAS), various galectins, S-opioid receptor ligands, cholecystokinin A receptor ligands, ligands specific for angiotensin AT1 or AT2 receptors, peroxisome proliferator-activated receptor γ ligands, β-lactam antibiotics, small organic molecules including antimicrobial drugs, and other molecules that bind specifically to a receptor preferentially expressed on the surface of targeted cells or on an infectious organism, or fragments of any of these molecules.

The phrase “a targeted receptor” refers to a receptor expressed by a cell that is capable of binding a cell targeting ligand. The receptor may be expressed on the surface of the cell. The receptor may be a transmembrane receptor. Examples of such targeted receptors include EGFR, αᵥβ₃ integrin, somatostatin receptor, folate receptor, prostate-specific membrane antigen, CD105, mannose receptor, estrogen receptor, GLUT1, LAT1, nicotinic acetylcholine receptors (nAChR), asialoglycoprotein receptor, and GM1 ganglioside.

Weakly basic groups useful in the silica nanoparticles may have a pKa between about 4.5 and about 7.0, e.g., a pKa of about 4.5, about 5, about 5.5, about 5.75, about 6, about 6.25, about 6.5, about 6.75, about 7, or a range between and including any two of the foregoing values, such as about 5.5 to about 7 or about 6 to about 7. In some embodiments, the weakly basic group is imidazole or pyridinyl. While not wishing to be bound by theory, it is expected that after uptake of SNPs into the cell by endocytosis, the SNP will reside in an endosome/lysosome vesicle. It is thought that weakly basic groups on the SNP can then be protonated in a “proton-sponge effect”, quickly leading to lysis of the endosome/lysosome and release of the SNP into the cytosol of the cell.

The present technology provides silica nanoparticles (SNPs) suitable for delivering water-soluble biomolecules into animal cells. In a first aspect, each nanoparticle includes a silica network comprising crosslinked polysiloxanes, wherein the crosslinks include disulfide linkages, the polysiloxanes optionally bear weakly basic functional groups, the nanoparticle comprises an exterior surface comprising surface-modifying groups attached to and surrounding the silica network, wherein the surface-modifying groups comprise PEG, polysarcosine, polyzwitterion or combinations of two or more thereof. The SNP may have an average diameter of 15 nm to 500 nm.

In a second aspect of the technology, the nanoparticle includes a silica network comprising crosslinked polysiloxanes, wherein the crosslinks include disulfide linkages, the polysiloxanes optionally bear weakly basic functional groups, the nanoparticle comprises an exterior surface comprising surface-modifying groups attached to and surrounding the silica network, wherein the surface-modifying groups comprise PEG, polysarcosine, polycation, polyanion, polyzwitterion or combinations of two or more of thereof. The SNP may have a surface potential ranging from −45 mV to +45 mV. The SNP may have an average diameter of 15 nm to 500 nm.

In a third aspect of the technology, the nanoparticle includes a silica network comprising crosslinked polysiloxanes, wherein the polysiloxanes may comprise siloxy subunits having the structure (I)

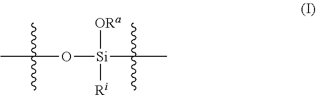

wherein
R^{a} at each occurrence is independently selected from a bond to a Si of another polysiloxane chain or a C₁₋₁₂ alkyl group;
R¹ at each occurrence is independently selected from the group consisting of C₁₋₁₂ alkyl and C₂₋₁₂ alkenyl groups, optionally substituted with a substituent selected from the group consisting of halogen and NR¹₂, wherein each occurrence of R¹ is independently selected from H or a C₁₋₁₂ alkyl group, or two R¹ groups, together with the N atom to which they are attached, form a pyrrolidine or piperidine ring;
the crosslinks between polysiloxanes comprise disulfide linkages,
the nanoparticle comprises an exterior surface comprising surface-modifying groups attached to and surrounding the silica network, wherein the surface-modifying groups comprise polyethylene glycol (PEG), polysarcosine, polyzwitterion, polycation, polyanion, or combinations of two or more thereof; and
the nanoparticle has an average diameter of 15 nm to 200 nm.

In any embodiments of the nanoparticle herein (which will be understood to include nanoparticles of any of the first, second and third aspects), the polysiloxanes may comprise a plurality of siloxy subunits having the structure (II) and/or the structure (III),

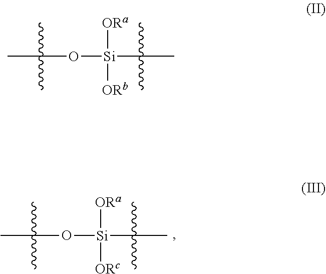

wherein R^{a} and R^{b} at each occurrence in the polysiloxane are independently selected from a bond to a Si of another polysiloxane chain or C₁₋₆ alkyl groups, and R^{c} is selected from C₂₋₆ alkenyl groups. In any embodiments, the polysiloxanes comprising the plurality of siloxy subunits having the structure (II)

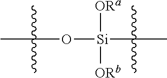

may include a first portion of siloxy subunits wherein R^{a} and R^{b} are independently selected from C₁₋₆ alkyl groups, and a second portion of siloxy subunits wherein one of R^{a} and R^{b} is independently selected from C₁₋₆ alkyl groups at each occurrence, and one of R^{a} and R^{b} is a bond to a Si of another polysiloxane chain. In any embodiments, the polysiloxanes comprising the plurality of siloxy subunits having the structure (II), may include a portion of the siloxy subunits wherein each of R^{a} and R^{b} is a bond to a Si of another polysiloxane chain. It will be appreciated that when R^{a} or R^{b} is a bond to a Si of another polysiloxane chain, the siloxysubunit is branched, forming a crosslink to another polysiloxane chain. In any embodiments, the plurality of siloxy subunits may be derived from tetraethoxysilane, i.e., these monomers are precursors which polymerize to form the siloxy subunits.

In any embodiment of any of the nanoparticles herein, including any siloxy subunits disclosed herein, such as, but not limited to siloxy subunits of structures (I), (II), or (III), R^{a} at each occurrence may be independently selected from a bond to a Si of another polysiloxane chain or a C₁₋₆ alkyl group.

In any embodiment of the present nanoparticles including a siloxy subunit of structure (I), Rⁱ at each occurrence may independently be selected from the group consisting of C₁₋₁₂ alkyl and C₂₋₁₂ alkenyl groups, optionally substituted with a substituent selected from the group consisting of halogen and NR¹₂, wherein each occurrence of R¹ is independently selected from H or a C₁₋₁₂ alkyl group.

In any embodiments, Rⁱ at each occurrence may be independently selected from the group consisting of C₁₋₁₂ alkyl and C₂₋₁₂ alkenyl groups, optionally substituted with a substituent selected from the group consisting of halogen and NR¹₂, wherein the two R¹ groups, together with the N atom to which they are attached, form a pyrrolidine or piperidine ring. In any embodiments, Rⁱ at each occurrence may be a C₁₋₄ alkyl group, optionally substituted with a halogen or NR¹₂ substituent. In any embodiments, Rⁱ at each occurrence may be a C₂₋₄ alkenyl group. In any embodiments, Rⁱ at each occurrence may be independently selected from methyl, propyl, 3-chloropropyl, 3-aminopropyl, 3-dimethylaminopropyl, and vinyl. In any embodiments, the siloxy subunits of structure (I) may be derived from one or more of triethoxymethylsilane, triethoxypropylsilane, triethoxy-3-chloropropylsilane, triethoxy-3-aminopropylsilane, triethoxy-3-dimethylaminopropylsilane, triethoxyoctylsilane, or triethoxyvinylsilane.

Silica nanoparticles of the present technology are multifunctional. The SNPs may include weakly basic groups, disulfide linkages, and/or surface-modifying groups. In any embodiments in which the weakly basic groups are present, they may include heteroaryl groups having a pka of about 4.5 to about 7.2, e.g., about 4.5, about 5, about 5.5, about 6, about 6.3, about 6.5, about 6.7, about 7, about 7.2 or a range between and including any two of the foregoing values. For example, the weakly basic groups may include imidazolyl, pyridinyl, picolinyl, lutidinyl, indolinyl, tetrahydroquinolinyl, or quinolinyl groups or a combination of two or more of the foregoing groups. In any embodiments, the weakly basic groups may include an imidazolyl group and/or pyridinyl group. In any embodiments, each weakly basic group is attached to a siloxy subunit and includes one of the following formulae (A, B, or C):

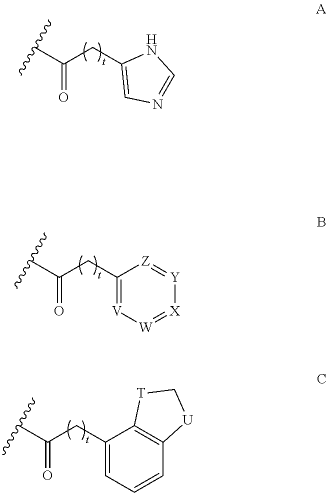

wherein

t at each occurrence is independently 0, 1, 2 or 3

one of T and U is NH and the other is CH₂;

one of V, W, X, Y, Z is N and the rest are selected from CH or CCH₃.

In any embodiments, the polysiloxanes may include siloxy subunits having the structures (IVA) or (IVB),

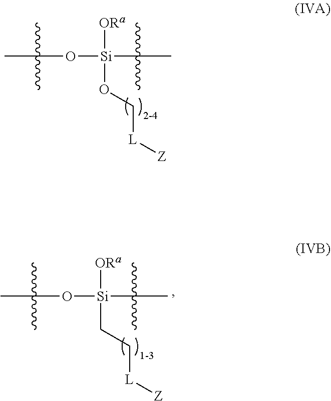

wherein
R^{a} at each occurrence is independently selected from C₁₋₆ alkyl groups or a bond to a Si of another polysiloxane chain;
L is a bond or is a linking group selected from —C(O)NH—, —O—, —NH—, —C(O)—, or —C(O)O; and
Z is at each occurrence independently a picolinyl, lutidinyl, indolinyl, tetrahydroquinolinyl, quinolinyl, imidazolyl, or pyridinyl group.

In any embodiments, the polysiloxanes may include siloxy subunits having the structure (IVB). In any embodiments, L may be —C(O)NH—. In any embodiments, Z may be imidazolyl. In any embodiments, the weakly basic groups may, e.g., comprise a siloxy subunit derived from N-(3-(triethoxysilyl)propyl)-1H-imidazole-2-carboxamide (TESPIC).

In any embodiments of the nanoparticles herein, the polysiloxanes may further include silyloxy subunits having structure (IVC):

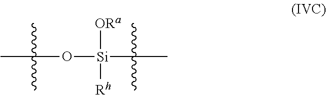

wherein
R^{a} at each occurrence is independently selected from a bond to a Si of another polysiloxane chain or a C₁₋₁₂ alkyl group; and
R^{h} at each occurrence is a moiety comprising a weakly basic group.

In any embodiments, the moiety of R^{h} may have a molecular weight of up to 300 Da and comprise any of the the weakly basic groups disclosed herein. For example, the weakly basic group of R^{h} may be selected from imidazolyl, pyridinyl, tetrahydroquinolinyl, or indolinyl groups, or a combination any two or more thereof. In any embodiments, R^{h} at each occurrence may independently have the structure —(CH₂)ₙ-L-Z, wherein L is a bond or is a linking group selected from —C(O)NH—, —O—, —NH—, —C(O)—, or —C(O)O; Z is at each occurrence is independently a picolinyl, lutidinyl, indolinyl, tetrahydroquinolinyl, quinolinyl, imidazolyl, or pyridinyl group; and n is 0, 1, 2, 3, or 4. In any embodiments, R^{h} may have the structure (D):

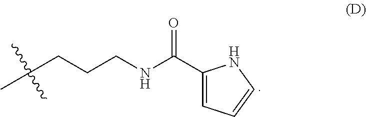 [SMILES: *CCCNC(=O)c1ccc[nH]1]

The polysiloxanes that make up the silica network are crosslinked, not only by siloxy linkages, but including by disulfide linkages. For example, the polysiloxanes may include a plurality of crosslinking siloxy subunits having the structure (V)

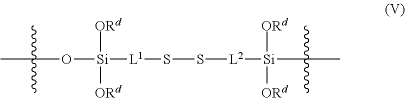

wherein L¹ and L² at each occurrence in the polysiloxanes are independently a C₁₋₆ alkylene group; R^{d} at each occurrence in the polysiloxanes is the same or different and is independently selected from a bond to another polysiloxane chain or C₁₋₆ alkyl groups. In any embodiments R^{d} may be the same at each occurrence, e.g., ethyl. In any embodiments, each of L¹ and L² may be ethylene, propylene, or butylene at each occurrence. In any embodiments, each of L¹ and L² may be propylene, at each occurrence. The disulfide bonds are sensitive to the levels of glutathione (GSH) naturally found in cells. While not wishing to be bound by theory, when SNPs enter a cell, the GSH in the cell is believed to reduce the disulfide bonds in the silica network, causing the silica network to fall apart and release any encapsulated water-soluble biomolecule into the cytosol of the cell.

SNPs of the present technology include an exterior surface comprising surface-modifying groups attached to and surrounding the silica network. The surface-modifying groups may comprise polyethylene glycol (PEG), polysarcosine, polyzwitterion, polycation, polyanion, or combinations of two or more thereof. In any embodiments, the surface-modifying groups may comprise polyethylene glycol (PEG), polysarcosine, polyzwitterion or combinations of two or more of thereof, or PEG, polysarcosine, polycation, polyanion, polyzwitterion or combinations of two or more of thereof. In any embodiments, the surface-modifying groups may include PEG and/or polysarcosine. The surface-modifying groups may further be conjugated to one or more of targeting ligands, biotin, CPP, imaging agents, or dyes.

PEG is a hydrophilic polymer comprising repeating ethylene oxide subunits and may be used as a surface-modifying group of the present SNPs. The PEG polymeric chains may be attached directly or through a linker to the polysiloxanes of the silica network. Each PEG terminates in one of various groups that, e.g., may be selected from a targeting ligand, OH, O—(C₁₋₆)alkyl, NH₂, CPP, biotin or a dye. In some embodiments the PEG terminates in OH or O—(C₁₋₆)alkyl, and in still others the PEG terminates in in an OC₁₋₃ alkyl group. In still other embodiments, the PEG terminates in a targeting ligand. The targeting ligand may be selected from the group consisting of a cofactor, carbohydrate, peptide, antibody, nanobody, or aptamer. For example, the targeting ligand maybe selected from the group consisting of glucose, RVG peptide, folic acid, mannose, GE11, cRGD, KE108, octreotide, PSMA aptamer, TRC105, 7D12 nanobody, all-trans retinoic acid (ATRA), 11-cis-retinal (11cRal), CTB, N-acetylgalactosamine (GalNAc) and combinations of two or more thereof. In other embodiments, the targeting ligand is selected from the group consisting of folic acid, mannose, GE11, cRGD, KE108, octreotide, TAT cell penetrating peptide, PSMA aptamer, TRC105, 7D12 nanobody, all-trans retinoic acid (ATRA), 11-cis-retinal (11cRal), CTB, and N-acetylgalactosamine (GalNAc). In any embodiments, the targeting ligand is selected from glucose, RVG peptide, or both.

Typically, each PEG chain has 23 to 340 repeat units or a molecular weight of about 1,000 to about 15,000 Da. Suitable molecular weights for each PEG chain on the SNP include about 1,000, about 1,500, about 2,000, about 2,500, about 3,000, about 4,000, about 5,0000, about 7,500, about 10,000, or about 15,000 Da, or a range between and including any two of the foregoing values (e.g., about 1,000 to about 10,000 Da or about 2,500 to about 7,500 Da).

In any embodiments of the SNP, the polysiloxanes may comprise a plurality of siloxy subunits having the structure (VI):

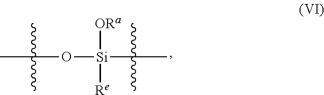

R^{a} (VI), wherein R^{a} at each occurrence is selected from a bond to Si from another polysiloxane chain or a C₁₋₆ alkyl group, and R^{e} at each occurrence is surface-modifying group, optionally including a C₁₋₆ linker group connecting the surface-modifying group to the Si atom to which R^{e} is attached. In certain embodiments, the C₁₋₆ linker group is present and connected to the surface-modifying group directly or via an amine, ether, amide, ester, urethane, urea, imine, or sulfide group. For example, the C₁₋₆ linker group may be —NHC(O)NH—(C₂₋₅ alkylene)-, —NHC(O)—(C₂₋₅ alkylene)-, —C(O)NH—(C₂₋₅ alkylene)-, —NH—(C₂₋₅ alkylene)-, —O—(C₂₋₅ alkylene)-, —S—(C₂₋₅ alkylene)-, —OC(O)NH—(C₂₋₅ alkylene)-, or —NHC(O)O—(C₂₋₅ alkylene)-. In any embodiments, the surface-modifying groups may comprise PEG attached to a siloxy subunit having the structure (VII):

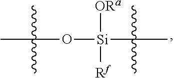

wherein R^{a} at each occurrence is selected from a bond to Si from another polysiloxane chain or a C₁₋₆ alkyl group, and R^{f} has the structure (E1) or (E2):

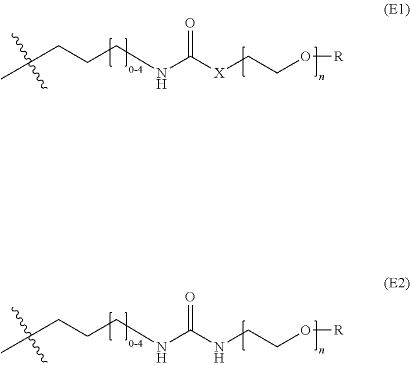

wherein X is O, NH, or CH₂O, and R is selected from the group consisting of H, a C₁₋₆ alkyl, targeting ligand, a cell-penetrating peptide (CPP), and an imaging agent. In any embodiments, the silica network may comprise two or more (e.g., 2, 3, 4, or 5) different siloxy subunits of structure (VII). For example in some embodiments, the silica network comprises siloxy subunits of structure (VII) wherein X is NH and R is a C₁₋₆ alkyl and siloxy subunits of structure (VII) wherein X is NH and R is a targeting ligand. In any embodiments, two or more distinct targeting ligands (e.g., glucose and RVG peptide) may be used (i.e., on two different siloxy subunits of structure (VII). In any embodiments, the surface-modifying groups may comprise PEG attached to a siloxy subunit having the structure, —O—Si(R^{g})₂—, wherein R^{g} at each occurrence is independently selected from OR^{a} or R^{f} as defined herein.

In any embodiments of any of the aspects of the present technology (including but not limited to the first, second or third aspects), the silica network of the SNPs comprises siloxy subunits having structure (I). In some such embodiments, the siloxy subunits having structure (I) comprise 1 to 80 mol % of the silica network, including for example, 1 mol %, 2 mol %, 5 mol %, 10 mol %, 15 mol %, 20 mol %, 25 mol %, 30 mol %, 35 mol %, 40 mol %, 45 mol %, 50 mol %, 55 mol %, 60 mol %, 65 mol %, 70 mol %, 75 mol %, or 80 mol % or a range between and including any two of the foregoing values. Additional examples of the present SNPs may therefore include from 10 mol % to 70 mol % or from 20 mol % to 60 mol %. In some embodiments of the first, second or third aspects of the present technology, no siloxy subunits of structures (II) or (III) are included in the silica network of the SNPs.

In any embodiments of any of the aspects of the present SNPs, the siloxy subunits having structures (II) or (III) comprise 1 to 80 mol % of the silica network, including for example, 1 mol %, 2 mol %, 5 mol %, 10 mol %, 15 mol %, 20 mol %, 25 mol %, 30 mol %, 35 mol %, 40 mol %, 45 mol %, 50 mol %, 55 mol %, 60 mol %, 65 mol %, 70 mol %, 75 mol %, or 80 mol % or a range between and including any two of the foregoing values.

In any embodiments of any of the aspects of the present SNPs, the molar percentage of disulfide-containing crosslinker (e.g., having the structure (V)) to the total siloxy subunits may range from 20 mol % to 80 mol %, including for example, 20 mol %, 30 mol %, 40 mol %, 50 mol %, 60 mol %, 70 mol %, 80 mol % or a range between and including any two of the foregoing values. The molar ratio of siloxy subunits bearing weakly basic groups (e.g., having any of structures (IVA), (IVB), or (NC)) to total siloxy subunits of the silica network may range from 0 mol % to 40 mol %, e.g., 0 mol %, 5 mol %, 10 mol %, 15 mol %, 20 mol %, 30 mol %, 40 mol % or a range between and including any two of the foregoing values. The molar ratio of siloxy subunits bearing surface modifying groups (e.g., having structure NA or IVB) to the total organosilica precursors may range from 10 mol % to 50 mol %, e.g., 10 mol %, 15 mol %, 20 mol %, 30 mol %, 40 mol %, or 50 mol % or a range between and including any two of the foregoing values.

In the present technology, the surface of the SNPs may also be charged (measured as zeta potential), so long as the net charge is not too great, e.g., −45 mV to +45 mV, preferably from −30 mV to +30 mV. Nanoparticle surface potential may be measured by DLS in an applied electric field at any suitable voltage (e.g., 40 V; the measured surface potential will be independent of the exact voltage used) at 0.1 mg/mL, pH 7.4, 25° C. Examples of the surface potential of the present SNPs include −45, −30, −25, −20, −15, −10, −5, +5, +15, +20, +25, +30, or +45 mV, or a range between and including any two of the foregoing values. Thus, e.g., the surface potential may be, e.g., −20 to +20 mV, −10 to +10, or −5 to +5 mV. In any embodiments, where the surface of the SNP bears charged functional groups, the net charge is or is about 0 mV, e.g., due to a polyzwitterion with an equal number of positively and negatively charged groups.

In the present technology, the surface of the SNPs may be charged due to the presence of surface-modifying groups that include ionizable functional groups on the SNP surface and/or in the SNP surface layer, provided the net charge is as described herein. For example, in any embodiments, the polysiloxanes of the silica network may comprise a plurality of siloxy subunits having the structure (IB):

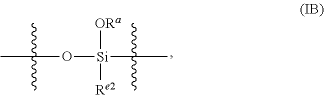

wherein R^{a} at each occurrence in the polysiloxane is a bond to Si from another polysiloxane chain or a C₁₋₆ alkyl group, and R^{e2} at each occurrence is a C₁₋₁₂ alkyl group, e.g., a C₁₋₆ alkyl group, substituted with a charged functional group. The charged functional groups may include positively and/or negatively charged functional groups, or ionizable functional groups that provide positively and/or negatively charged groups.

In any embodiments, the surface-modifying groups may include positively charged functional groups. In any embodiments, the positively charged functional groups may include an ionizable group selected from amine, amidine, guanidine, pyridinyl or combinations of two or more thereof. For example, R^{e2} may be an amino-(C₂₋₄ alkyl) group such as an amino propyl group. The surface-modifying groups may also include a cationic polymer or CPP. For example, the cationic polymer may be selected from the group consisting of polyethyleneimine (PEI), polylysine, polyarginine, and polyamidoamine (PAMAM). In any embodiments, the CPP may be selected from any of those disclosed herein.

In any embodiments, the surface-modifying groups may include negatively charged groups. In any embodiments, the negatively charged groups may include ionizable functional groups selected from carboxyl, sulfonyl, sulfate, phosphate, or combinations thereof. In any embodiments, R^{e} may be a carboxyl-(C₂₋₄ alkyl) group. The surface-modifying groups may also include an anionic polymer. In any embodiments, the anionic polymer may be selected from the group consisting of poly(glutamic acid) and poly(acrylic acid).

In any embodiments, the surface-modifying groups may include positively charged functional groups and negatively charged groups, i.e., a polyzwitterion. The polyzwitterion may include any combination of the positively and negatively charged groups disclosed herein. In any embodiments, the surface-modifying group may be a polyzwitterion selected from poly(carboxybetaine methacrylate) (PCBMA). poly(sulfobetaine methacrylate) (PSBMA), poly(2-methacryloyloxyethyl phosphorylcholine) (PMPC), and combinations of two or more thereof.

In any embodiments, where the surface-modifying groups include a charged polymer (e.g., polyzwitterion, polycation or polyanion), the polymer may have a Mn of about 1,000 to about 50,000 Da. For example, the polyzwitterion, polycation or polyanion may have a Mn of about 1,000, about 2,000, about 3,000, about, 4,000, about 5,000, about 7,500, about 10,000, about 15,000, about 20,000, about 30,000, about 40,000, about 50,000 Da or a value within a range between and including any two of the foregoing values. For example, the polyzwitterion, polycation or polyanion may have a Mn of about 2,000 to about 10,000 Da.

The present SNPs may be roughly sphere-shaped or may have a more elongated shape. Nevertheless, the “average diameter” of the present SNPs means the average hydrodynamic diameter and ranges from 15 nm to 500 nm. Thus, the present SNPs may have an average hydrodynamic diameter of 15, 20, 30, 40, 50, 60, 70, 80, 90, 100, 110, 125, 150, 175, 200, 300, 400, or 500 nm or a range between and including any two of the foregoing values. In any embodiments herein, they may have an average hydrodynamic diameter of 20 to 150 nm or even 20 nm to 100 nm, or 20 nm to 20 nm.

In any embodiments, the present SNPs further include a water-soluble biomolecule non-covalently bound to (e.g., carried by) the nanoparticle. For example, the water-soluble biomolecule may be encapsulated by the SNP and/or electrostatically bound to the SNP. In any embodiments the majority (>50 mol %) of the water-soluble biomolecule is encapsulated within the SNP. As used herein, “water-soluble” refers to a solubility of at least 1 mg/ml in water at pH 7 and 25° C. The water-soluble biomolecules of the present technology (also referred to as “biomacromolecule” herein) may be a polynucleic acid, polypeptide (including proteins), or a polynucleic acid/polypeptide complex, e.g., DNA, RNA, an enzyme, or a ribonucleoprotein complex (RNP). They may be naturally occurring or unnatural; that is they may be isoloated from their natural sources or may be chemically synthesized or produced via standard techniques of biotechnology such as site-directed mutagenesis, cloning, or the like. In any embodiments, the water-soluble biomolecule may be selected from the group consisting of plasmid DNA (pDNA), single-stranded donor oligonucleotide (ssODN), complementary (cDNA), messenger RNA (mRNA), small interfering RNA (siRNA), microRNA (miRNA), short hairpin RNA (shRNA), single guide RNA (sgRNA), transfer RNA (tRNA), ribozymes, and combinations of two or more thereof (e.g., mRNA and sgRNA, Cas9 mRNA and sgRNA). In certain embodiments, the water-soluble biomolecule may be selected from the group consisting of Cas9 RNP, RNP+ssODN where ssODN serves as a repair template, RNP+donor DNA up to 2 kb, other Cas9-based protein/nucleic acid complexes, and base editors, e.g., cytosine base editors (CBE), adenine base editors (ABE). It will be appreciated that with the present nanoparticles, Cas9 or RNP need not be conjugated to any repair template as either may simply be mixed with the desired polynucleic acid instead during the nanoparticle formation process. NLS peptides may be used to direct water-soluble biomolecule to the nucleus if desired. For example, polynucleic acids as described herein as well as proteins such as Cas9 or RNP+ donor DNA complexes may be covalently tagged (i.e., conjugated) with NLS peptides using techniques well known in the art. DNA

The present SNPs may have a biomolecule loading content of from about 1 wt % to about 20 wt %, e.g., about 1 wt %, about 2 wt %, about 3 wt %, about 4 wt %, about 5 wt %, about 6 wt %, about 7 wt %, about 8 wt %, about 9 wt %, about 10 wt %, about 12 wt %, about 14 wt %, about 15 wt %, about 16 wt %, about 18 wt %, or about 20 wt %, or a range between and including any two of the foregoing values. Thus, in any embodiments, the biomolecule loading content of the SNP may be, e.g., from about 2 wt % to 20 wt %, about 5 wt % to about 15 wt %, or about 8 or 9 wt % to about 10 wt %. Loading efficiency of the present SNPs with biomolecules is high. In any embodiments, the loading efficiency may be greater than 80%, greater than 85%, or even greater than 90%, e.g., 80%, 85%, 90%, 95%, 99% or a range between and including any two of the foregoing values.

In any embodiments, the water-soluble biomolecule may be tagged with an imaging agent, e.g., a dye as described herein. Alternatively, an imaging agent may be attached to the organosilica network. The imaging agent (e.g., dye) may be attached to the organosilica network via bonds to amino groups in the organosilica network. By way of a non-limiting example, the bonds may be amide bonds, N—C bonds, imino bonds and the like.

In another aspect, the present technology provides methods of making the silica nanoparticles described herein. The methods include forming a nanoparticle comprising an organosilica network as described herein by combining an aqueous solution, optionally containing the water-soluble biomolecules and a solution of organosilica network precursors (including any of those described herein, such as those bearing disulfide crosslinks and those bearing weakly basic groups) in an immiscible organic solvent, and forming an emulsion, e.g., by rapid stirring. Optionally, a catalyst such as a base is added to facilitate the polymerization of the organosilica network precursors to form the organosilica network. After the initial polymerization, siloxy precursors with surface-modifying groups (e.g., PEG, polysarcosine, polyzwitterion, polycation, polyanion, or combinations of two or more thereof) may be added to the mixture to polymerize with the nascent nanoparticles and provide the uncharged or low-surface potential exterior surface of the SNP. The precursors to the surface-modifying groups may be further functionalized (e.g., with targeting ligands, CPP, imaging agents, etc.) before or after being added to the nanoparticle mixture.

The organosilica network precursors may include various tetraalkoxysilanes and organosiloxy disulfide monomers. Tetralkoxysilanes may be referred to as 4-arm precursors to siloxy subunits (e.g., the siloxy subunit of structure (II)) that can form up to 4 siloxy linkages in the silica network the present SNPs. Trialkoxy alkyl silanes or trialkoxy alkenyl silanes may be used in place of or in addition to the tetraalkoxysilane. Thus, trialkoxy alkyl silanes or trialkoxy alkenyl silanes may be referred to as 3-arm precursors to siloxy subunits (e.g., the siloxy subunit of structure (I)) that can form up to 3 siloxy linkages in the silica network of the present SNPs. The alkyl group of the trialkoxy alkyl silanes may include the weakly basic groups. The water-soluble biomolecule may selected from any of the biomolecules disclosed herein. The emulsion may be formed from any suitable organic solvents (including, e.g., alkanes, cycloalkanes, alcohols and non-ionic detergents and mixtures of any two or more thereof) and water. In any embodiment, the emulsion may include hexanol, cyclohexane, Triton X-100 (polyethylene glycol p-(1,1,3,3-tetramethylbutyl)-phenyl ether) and water. In any embodiments, the emulsion may be formed by any suitable methods such as rapid stirring, shaking, vortexing, and sonication. The emulsion must be agitated sufficiently vigorously to form nanoparticles of the size desired for the present technology, e.g., ≤500 nm, preferably 20-100 nm, when carrying the water-soluble biomolecule. The molar ratio of disulfide-containing crosslinker to the total organosilica precursors may range from 20 mol % to 80 mol %, including for example, 20 mol %, 30 mol %, 40 mol %, 50 mol %, 60 mol %, 70 mol %, 80 mol % or a range between and including any two of the foregoing values. The molar ratio of siloxy precursors bearing weakly basic groups as described herein may range from 0 mol % to 40 mol %, e.g., 0 mol %, 5 mol %, 10 mol %, 20 mol %, 30 mol %, 40 mol % or a range between and including any two of the foregoing values. The molar ratio of siloxy precursors bearing surface modifying groups to the total organosilica precursors may range from 10 mol % to 50 mol %, e.g., 10, 20, 30, 40, or 50 mol % or a range between and including any two of the foregoing values. The surface modifying groups used may have one or more targeting ligands, CPP, biotin, or imaging agents (such as dyes) attached before the surface modifying groups are incorporated into the present SNPs. Alternatively, the targeting ligands, CPP, biotin and imaging agents may be attached to the surface-modifying groups after those groups are incorporated onto the SNP.

In any embodiments, the present methods may further include attaching one or more of a targeting ligand, a CPP, biotin, or an imaging agent to the surface of the SNP. The targeting ligands and other groups to be attached typically have a reactive group such as an electrophile or active ester or the like which can react with, e.g., a nucleophilic group on the organosilica network or surface-modifying group such as, but not limited to amino groups. Other amide-bond forming methods or click chemistry may be used join the targeting ligand, CPP, biotin or imaging agent to the nanoparticle. Alternatively, the CPP, and charged groups, including surface-modifying groups such as the polycation, polyzwitterion or polyanion surface-modifying groups can simply be adsorbed to the surface of the nanoparticle via electrostatic interactions. The nanoparticles thus formed may be precipitated from solution with a suitable organic solvent, e.g., acetone.

In another aspect, the present technology provides methods of delivering a water-soluble biomolecule to a target cell for any suitable purpose, e.g., gene editing, gene silencing, therapy, etc. The methods include exposing the targeted cell to an effective amount of any of the herein-described nanoparticles. By an effective amount is meant an amount sufficient to produce a detectable or measurable amount of infiltration of the SNP into the target cell and/or produce a detectable or measurable effect in said cell. The methods include both in vitro and in vivo methods. For example, the methods may include exposing an effective amount of any of the herein-described nanoparticles to tissue culture. In any embodiments, the cell may be exposed to the SNP via any route of administration described herein. In any embodiments, the water-soluble biomolecule is any of those described herein, including but not limited to DNA, pDNA, mRNA, siRNA, Cas9 RNP, RNP+donor nucleic acids.

In another aspect, the present technology provides methods of treating a condition or disorder in a subject that may be ameliorated by any of the types of biomolecules disclosed herein. In any embodiments, the methods include administering to the subject an effective amount of a nanoparticle including a biomolecule as as disclosed herein, i.e., a therapeutically effective amount to ameliorate or cure the condition or disorder. For example, the methods may include administering any of the herein-described nanoparticles to a subject in need thereof (i.e., a subject in need of the biomolecule to be delivered by the nanoparticle). As used herein, a “subject” is a mammal, such as a cat, dog, rodent or primate. In some embodiments, the subject is a human. In some embodiments, the payload is any of those described herein, including but not limited to pDNA, mRNA, siRNA, Cas9 RNP, or Simplex. In any embodiments of the method, the condition or disorder occurs in the central nervous system of the subject, and the nanoparticle comprises glucose and/or RVG peptide targeting ligands.

The compositions described herein can be formulated for various routes of administration, for example, by parenteral, intravitreal, intrathecal, intracerebroventricular, rectal, nasal, vaginal administration, direct injection into the target organ, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not degrade the SNPs described herein. Injectable forms may be prepared with acceptable solvents or vehicles including, but not limited to sterilized water, phosphate buffer solution, Ringer's solution, 5% dextrose, or an isotonic aqueous saline solution.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Exemplary carriers and excipients may include but are not limited to USP sterile water, saline, buffers (e.g., phosphate, bicarbonate, etc.), tonicity agents (e.g., glycerol),

Specific dosages may be adjusted depending on conditions of disease, the age, body weight, general health conditions, sex, and diet of the subject, dose intervals, administration routes, excretion rate, and combinations of drug conjugates. Any of the above dosage forms containing effective amounts are well within the bounds of routine experimentation and therefore, well within the scope of the instant present technology. By way of example only, such dosages may be used to administer effective amounts of the present SNPs (loaded with a biomolecule) to the patient and may include 0.1, 0.2, 0.3, 0.4, 0.5, 0.75, 1.0, 1.5, 2.0, 2.5, 3.0, 4.0, 5.0, 6.0, 7.0, 8.0, 9.0, 10, 11, 12, 13, 14, 15 mg/kg or a range between and including any two of the forgoing values such as 0.1 to 15 mg/kg. Such amounts may be administered parenterally as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once per day, per 2 days, per 3 days, per week, per 10 days, per 2 weeks, or a range between and including any of the foregoing frequencies. Alternatively, the compositions may be administered once per day on 2, 3, 4, 5, 6 or 7 consecutive days. A complete regimen may thus be completed in only a few days or over the course of 1, 2, 3, 4 or more weeks.

The examples herein are provided to illustrate advantages of the present technology and to further assist a person of ordinary skill in the art with preparing or using the nanoparticles compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations or aspects of the present technology described above. The variations or aspects described above may also further each include or incorporate the variations of any or all other variations or aspects of the present technology.

### EXAMPLES

### Materials and General Procedures

Materials and Instrumentation. Tetraethyl orthosilicate (TEOS), 1H-imidazole-4-carboxylic acid, thionyl chloride (SOCl₂), Triton X-100, acetone, ethanol, glutathione (GSH), trifluoroacetate (TFA), 1-ethyl-3-(3-dimethylaminopropyl)carbodiimide (EDC), 1-hydroxybenzotriazole hydrate (HOBt), N-hydroxysuccinimide (NHS), tris(2-carboxyethyl)phosphine hydrochloride (TCEP) and ammonia (30% in water) were purchased from Fisher Scientific, USA. Hexanol, cyclohexane, and (3-aminopropyl)triethoxysilane (APTES), were bought from Tokyo Chemical Industry Co., Ltd., USA. Triethylamine (TEA) and dimethyl sulfoxide (DMSO) were purchased from Alfa Aesar, USA. Bis[3-(triethoxysilyl)propyl]-disulfide (BTPD) was purchased from Gelest, Inc., USA. Methoxy-poly(ethylene glycol)-silane (mPEG-silane, Mₙ=5000), amine-poly(ethylene glycol)-silane (NH₂-PEG-silane, Mₙ=5000) and Maleimide-poly(ethylene glycol)-silane (Mal-PEG-silane, Mₙ=5000), were purchased from Biochempeg Scientific Inc., USA. All-trans-retinoic acid (ATRA) was purchased from Santa Cruz Biotechnology, USA. A cell penetrating peptide TAT (sequence: CYGRKKRRQRRR) was purchased from GenScript Biotech Corporation, USA. Methoxy-poly(ethylene glycol)-silane (mPEG-silane, Mₙ=5000) and maleimide-poly(ethylene glycol)-silane (Mal-PEG-silane, Mₙ=5000) were also purchased from JenKem Technology, USA. Carboxylate-poly(ethylene glycol)-silane (HOOC-PEG-silane, Mₙ=5000) was purchased from Nanocs Inc., USA. 1,2-O-isopopylidene-α-D-glucofuranoside was purchased from Santa Cruz Biotechnology, USA. RVG peptide with a C-terminal cysteine (sequence: YTIWMPENPRPGTPCDIFTNSRGKRASNGC) was purchased from GenScript Biotech Corporation, USA. Nuclear localization signal (NLS)-tagged Streptococcus pyogenes Cas9 nuclease (sNLS-SpCas9-sNLS) was obtained from Aldevron, USA. Single guide RNAs (sgRNAs) were purchased from Integrated DNA Technologies Inc., USA. Nuclear magnetic resonance (NMR) spectroscopy was performed on an Avance 400 (Bruker Corporation, USA).

SNP Characterization Techniques. The hydrodynamic diameters and zeta potentials of the SNPs were characterized by a dynamic light scattering (DLS) spectrometer (Malvern Zetasizer Nano ZS) at a 90° detection angle with a sample concentration at 0.1 mg/mL and pH of 7.4 at 25° C. To calculate the loading content and loading efficiency of the payloads in the SNPs, SNPs were re-suspended in water (1 mg/mL, 40 μL) and incubated with 0.1 M GSH aqueous solution (pH 7.4, 160 μL) with pH 7.4 for 1 h to allow for complete release of the payload. The RNP loading contents and loading efficiencies were measured via a bicinchoninic acid assay (BCA assay, Thermo Fisher, USA). DNA and mRNA loading contents and loading efficiencies were quantified using a NanoDrop One (Thermo Fisher, USA) by measuring OD₂₆₀.

Cell Culture for In Vitro Studies. Human embryonic kidney cells (i.e., HEK293 cells) were used for in vitro studies. HEK293 cells were purchased from ATCC. Green fluorescence protein (GFP)-expressing HEK 293 cells were bought from GenTarget Inc. Blue fluorescence protein (BFP)-expressing HEK 293 cells generated through lentiviral transduction of a BFP dest clone was obtained from Addgene. All HEK 293 cells were cultured with DMEM medium (Gibco, USA) added with 10% (v/v) fetal bovine serum (FBS, Gibco, USA) and 1% (v/v) penicillin-streptomycin (Gibco, USA). Cells were cultured in an incubator (Thermo Fisher, USA) at 37° C. with 5% carbon dioxide at 100% humidity

DNA and mRNA Transfection Efficiency Study. A red fluorescence protein (RFP)-expressing plasmid DNA (i.e., RFP-DNA, Addgene #40260, USA) and an RFP-mRNA (Trilink Biotechnologies #L-7203, USA) were used for DNA and mRNA transfection studies, respectively. HEK293 cells were placed into 96-well plates 24 h prior to treatment, at a density of 12,000-15,000 cells/well. Cells were incubated with either RFP-DNA-loaded SNPs, or RFP-mRNA-loaded SNPs. A commercially available transfection agent, Lipofectamine 2000 (Lipo 2000), was used as the positive control. The dosage of DNA or mRNA was 200 ng/well. The Lipo 2000-DNA (or Lipo 2000-mRNA) complex was prepared following the protocols of the manufacturer, with a final dosage of Lipo 2000 at 0.5 μL per well. Untransfected cells were used as the negative control. After 48 h, cells were harvested with 0.25% trypsin-EDTA, spun down and resuspended in 500 μL PBS. RFP expression efficiencies were obtained with a flow cytometer and analyzed with FlowJo 7.6.

RNP Genome-Editing Efficiency Study. For gene deletion studies, GFP-expressing HEK 293 cells were used as an RNP delivery cell model. RNP was prepared by mixing sNLS-SpCas9-sNLS and in vitro transcribed sgRNA (GFP protospacer: 5′-GCACGGGCAGCTTGCCGG-3′) at 1:1 in molar ratio. Cells were seeded at a density of 5,000 cells per well onto a 96-well plate 24 h before treatment. Cells were treated with RNP-loaded SNPs or RNP-complexed Lipo 2000 (0.5 μL/well). For each treatment, the RNP dosage was kept at 150 ng/well, with an equivalent Cas9 protein dosage at 125 ng/well.

For gene correction studies, BFP-expressing HEK 293 cells were employed as a model cell line. The RNP+ssODN mixture was prepared by simply mixing the as-prepared BFP gene-targeting RNP (BFP protospacer: 5′-GCTGAAGCACTGCACGCCAT-3′) and single-stranded oligonucleotide DNA (ssODN) (BFP to GFP ssODN sequence: 5′-CCTGAAGTTCATCTGCACCACCGGCAAGCTGCCCGTGCCCTGGCCCACCCTCGTGAC CACCCTGACGTACGGCGTGCAGTGCTTCAGCCGCTACCCCGACCACATGA-3′, changing BFP to GFP via alternation of histidine to tyrosine) donor template at 4° C. for 5 min at a 1:1 molar ratio. When editing correction (i.e., gene knock-in) occurs, three nucleotides within the BFP gene will be converted to a green fluorescent protein (GFP) gene, and thus the percentage of GFP positive cells can be used to evaluate the genome editing efficiency. BFP-expressing HEK 293 cells were seeded at a density of 5,000 cells per well onto a 96-well plate 24 h before treatment. Cells were treated with RNP+ssODN-loaded SNPs or Lipo 2000 (0.5 μL/well) carrying RNP+ssODN as the positive control. For each treatment, the RNP+dosage was kept at 150 ng/well (i.e., an equivalent Cas9 protein dosage of 125 ng/well), and the ssODN dosage was 25 ng/well.

The precise genome editing efficiencies were quantified six days after treatment using flow cytometry by counting the percentage of green fluorescence positive cells.

Cell Viability Assay. The cytotoxicity of SNPs was studied by an MTT assay. Cells were treated with complete medium, DNA-complexed Lipo 2000 (0.5 μL/well), and DNA-loaded SNPs, with concentrations ranging from 10 to 1000 μg/mL. Cell viability was measured using a standard MTT assay 48 h after treatment (Thermo Fisher, USA). Briefly, cells were treated with media containing 500 μg/mL MTT and incubated for 4 h. Then, the MTT-containing media was aspirated, and the purple precipitate was dissolved in 150 μL of DMSO. The absorbance at 560 nm was obtained with a microplate reader (GloMax® Multi Detection System, Promega, USA).

Subretinal Injection. All animal research was approved by UW-Madison animal care and use committee. Ai14 reporter mice (obtained from The Jackson Laboratory) were used to assess the mRNA delivery/genome editing efficiency induced by mRNA- or RNP-encapsulated SNP-PEG-ATRA, respectively. Cre-mRNA was purchased from Trilink Biotechnologies, USA (#L-7211). RNPs were prepared using either a sgRNA targeting the stop cassette composed of 3× SV40 polyA blocks (protospacer: 5′-AAGTAAAACCTCTACAAATG-3′) in Ai14 mice, or a mouse negative control sgRNA (Integrated DNA Technologies). Subretinal injection and subsequent RPE tissue collection were performed as reported previously. Mice were maintained under tightly controlled temperature (23±5° C.), humidity (40-50%), and light/dark (12/12 h) cycle conditions under a 200-lux light environment. The mice were anesthetized by intraperitoneal injection of ketamine (80 mg/kg), xylazine (16 mg/kg) and acepromazine (5 mg/kg) cocktail. Subretinal injection was performed as previously reported. For mRNA delivery studies, right eyes of mice were injected with mRNA-encapsulated SNP-PEG-ATRA (2 ul with 4 μg mRNA), and left eyes were injected with PBS. For RNP delivery studies, right eyes of mice were injected with SNP-PEG-ATRA encapsulating RNP with a sgRNA targeting the Ai14 stop cassette (i.e., Ai14 SNP), left eyes of mice were injected with SNP-PEG-ATRA encapsulating RNP with a negative control sgRNA (i.e., negative control SNP). The injection volume was 2 ul, containing 4 ug RNP. SNP-PEG-ATRA was injected into the subretinal space using a UMP3 ultramicro pump fitted with a NanoFil syringe, and the RPE-KIT (all from World Precision Instruments, Sarasota, Fla.) equipped with a 34-gauge beveled needle. The tip of the needle remained in the bleb for 10 s after bleb formation, then it was gently withdrawn.

Collected eyes were rinsed twice with PBS and puncture was made at or a serrata with an 18-gauge needle. The eye was opened along the corneal incisions and the eyecup was incised radially to the center and flattened to give a final floret shape. The RPE layer was then separated and flat-mounted on a cover-glass slide (i.e., RPE floret). RPE florets were imaged with a NIS-Elements using a Nikon C2 confocal microscope.

Intravenous Injection. Method A: Ai14 mice (6-8 weeks; three mice in each group) were injected with Cre-mRNA (20 μg per mouse) or RNP (100 μg per mouse)-encapsulated SNP-PEG or SNP-PEG-GalNAc through retro-orbital injections; PBS injected Ai14 mice were used as controls. The SNP-injected and control mice were sacrificed 3 days (Cre mRNA) or 7 days (RNP) post-injection. Organs and tissues (liver, heart, lung, spleen, kidney and muscle) were then collected and analyzed.

Fresh organs/tissues were imaged using the in vivo imaging system (IVIS Lumina system, Perkin Elmer) for tdTomato expression. A portion of liver samples were weighed and homogenized with cell lysis buffer as reported previously. See Z. He, Y. Hu, T. Nie, H. Tang, J. Zhu, K. Chen, L. Liu, K. W. Leong, Y. Chen, H.-Q. Mao, Size-controlled lipid nanoparticle production using turbulent mixing to enhance oral DNA delivery, Acta biomaterialia, 81 (2018) 195-207. The homogenized liver samples were added to 96-well black/clear flat bottom Imaging Microplate (Corning Life Science, USA), the tdTomato fluorescence was measured and analyzed by the IVIS system.

Intravenous Injection. Method B: Ai14 mice (6-8 weeks) were fasted for 24 h, and injected with Cre-mRNA (2 mg/kg) or Cas9 RNP (5 mg/kg)-encapsulated SNPs through retro-orbital injections; PBS injected Ai14 mice were used as controls. Thirty minutes post injection, blood glucose was restored by intraperitoneal injection of 200 μl of 20 wt % D-(+)-glucose solution in 1×PBS. The SNP-injected and control mice were perfused with ice cold PBS 14 days post-injection. Organs and tissues (brain, liver, heart, lung, spleen, kidneys, and muscle) were then collected and analyzed. Fresh organs/tissues were imaged using the in vivo imaging system (MS Lumina system, Perkin Elmer) for tdTomato expression.

FACS Analysis of Edited Cell Types in the Brain. The brain cells were dissociated and collected following previously established protocols with minor changes. (F. J. Rubio, X. Li, Q.-R. Liu, R. Cimbro, B. T. Hope, Fluorescence activated cell sorting (FACS) and gene expression analysis of Fos-expressing neurons from fresh and frozen rat brain tissue, JoVE (Journal of Visualized Experiments), (2016) e54358; E. E. Crouch, F. Doetsch, FACS isolation of endothelial cells and pericytes from mouse brain microregions, Nature Protocols, 13 (2018) 738-751.) Briefly, after PBS perfusion and tissue collection, brains were cut into 1 mm pieces coronally using a coronal mouse brain matrix (CellPoint Scientific, USA) on ice. The brain pieces from mid-brain were mixed with 50 μL Hibernate A low fluorescence buffer (Brain Bits, USA) and thoroughly mince on a glass slide on ice. The minced tissues were transferred into 1.5 mL Hibernate A low fluorescence buffer and precipitated by centrifugation at 110×g for 4 min at 4° C. The pellet was then mixed with 1.5 mL cold Accutase cell detachment solution (ThermoFisher, USA) for 30 min at 4° C. before centrifugation and resuspension in Hibernate A low fluorescence buffer. The digested tissue was mechanically triturated and fixed by 50% ethanol at 4° C. The cell suspension was re-dispersed in Hibernate A low fluorescence buffer and filtered through cell strainers (100 μm and 40 μm) before immunofluorescence staining.

Immunofluorescence Staining. Tissues were fixed in 4% paraformaldehyde (PFA) at RT for 24 hours, then switched to PBS solution containing 30% sucrose and stored at 4° C. for 72 h. Thereafter, the tissues were embedded in Tissue-Tek® Optimal Cutting Temperature Compound (Sakura Finetek, USA), and frozen in dry ice. The blocks were sectioned using a cryostat machine (CM1900, Leica Biosystems, USA) at 8 μm thickness and mounted on microscope slides. The sections were incubated in 10% goat serum and 0.3% Trixon X-100 in PBS at RT for 1 h. For immunofluorescence staining, the sections were first incubated with a rabbit anti-tdTomato primary antibody (ab152123, 1:5000, Abcam, USA) for 1 h at RT. The primary antibody was then detected by a fluorescence-conjugated secondary antibody (goat anti-rabbit IgG H&L (Alexa Fluor® 594), ab150080, 1:1000, Abcam, USA). Finally, the slides were mounted with DAPI and covered with microscope cover glasses. All of the images were acquired using CLSM.

For FACS analysis, brain cells were incubated in 10% goat serum and 0.3% Trixon X 100 in PBS at RT for 1 h. The cell samples were then separated into 3 equal aliquots for cell marker/RFP primary antibody (1 h) and corresponding secondary antibody (1 h) staining as shown below:

NeuN+ cells (neurons):

RFP: Rabbit Anti-RFP (ab152123, 1:5000)+ Anti-Rabbit IgG H&L (Alexa Fluor®594) (ab150080, 1:1000)

Neurons: Mouse anti-NeuN (ab104224, 1:1000)+ Anti-Mouse IgG H&L (Alexa Fluor® 488) (ab150113, 1:1000)

GFAP+ cells (astrocytes):

RFP: Rabbit Anti-RFP (ab152123, 1:5000)+ Anti-Rabbit IgG H&L (Alexa Fluor®594) (ab150080, 1:1000)

Astrocytes: Mouse anti-GFAP (ab104224, 1:1000)+ Anti-Mouse IgG H&L (Alexa Fluor® 488) (ab150113, 1:1000)

CD31+ cells (BCECs):

RFP: Rabbit Anti-RFP (ab152123, 1:5000)+ Anti-Rabbit IgG H&L (Alexa Fluor®594) (ab150080, 1:1000)

BCECs: Rat anti-CD31 (ab56299, 1:400)+ Anti-Rat IgG H&L (Alexa Fluor® 647) (ab150113, 1:500)

After antibody staining, the cell samples are finally mounted with DAPI and resuspended in cold PBS for storage and FACS analysis.

For CLSM imaging, tissues were fixed in 4% paraformaldehyde (PFA) at RT for 24 h, then switched to PBS solution containing 30% sucrose and stored at 4° C. for 72 h. Thereafter, the tissues were embedded in Tissue-Tek® Optimal Cutting Temperature Compound (Sakura Finetek, USA), and frozen in dry ice. The blocks were sectioned using a cryostat machine (CM1900, Leica Biosystems, USA) at 8 μm thickness and mounted on microscope slides. The sections were incubated in 10% goat serum and 0.3% Trixon X-100 in PBS for 1 h. For immunofluorescence staining, the sections were first incubated with corresponding primary antibodies for 1 h at room temperature. The primary antibody was then detected by a fluorescence-conjugated secondary antibody. Finally, the slides were mounted with DAPI and covered with microscope cover glasses. All the images were acquired using CLSM. The primary and secondary antibody sets are shown below:

RFP: Rabbit Anti-RFP (ab152123, 1:5000)+ Anti-Rabbit IgG H&L (Alexa Fluor®594) (ab150080, 1:1000)

Neurons: Mouse anti-NeuN (ab104224, 1:1000)+ Anti-Mouse IgG H&L (Alexa Fluor® 488) (ab150113, 1:1000)

BCECs: Rat anti-CD31 (ab56299, 1:400)+ Anti-Rat IgG H&L (Alexa Fluor® 647) (ab150113, 1:500)

Blood Biochemical Profile. Blood samples were immediately collected from the orbital sinus of each mouse from the SNP-treated groups or PBS control groups and centrifuged at 1500 g and 4° C. for 10 min for serum preparation. Clinical biochemical assessment of levels of blood urea nitrogen (BUN), creatinine (CRE), alanine aminotransferase (ALT), aspartate aminotransferase (AST), alkaline phosphatase (ALP), total bilirubin (TBIL), glucose (GLU), Calcium (CA), total protein (TP), albumin (ALB), globulin (GLOB), Na⁺, K⁺, Cl⁻ and total carbon dioxide (tCO2) was performed using VetScan Preventative Care Profile Plus rotors (Abaxis, USA) in a VetScan VS2 chemistry analyzer (Abaxis, USA).

Statistical Analysis. Results are presented as mean±standard deviation (SD). One-way analysis of variance (ANOVA) with Tukey's multiple comparisons was used to determine the difference between independent groups. Statistical analyses were conducted using GraphPad Prism software versions 6 and 8.

### Example 1—Synthesis of N-(3-(Triethoxysilyl)Propyl)-1H-Imidazole-4-Carboxamide (TESPIC)

A mixture of 1H-imidazole-4-carboxylic acid (250 mg, 1.9 mmol) and SOCl₂ (4 mL) was refluxed at 75° C. overnight. The reaction mixture was then cooled down to room temperature and added into 20 mL anhydrous toluene. The precipitate was collected by filtration and vacuum-dried to yield the intermediate, 1H-imidazole-4-carbonyl chloride. The as-prepared 1H-imidazole-4-carbonyl chloride was suspended in anhydrous THE (5 mL), followed by the addition of triethylamine (232 mg, 2.3 mmol) and APTES (420 mg, 1.9 mmol). The mixture was stirred at room temperature overnight under a nitrogen atmosphere, and then filtered. The solvent was removed by rotary evaporation to yield the final product TESPIC. Since the silica reactants have the tendency to undergo hydrolysis/polymerization during column purification, TESPIC was synthesized and used without purification. ¹H NMR (400 MHz, DMSO-D6): δ 0.62 (dd, 2H, J=14.6, 6.2 Hz), δ 1.12 (t, 9H, J=7.0 Hz), δ 1.60 (dt, 2H, J=15.9, 8.0 Hz), δ 2.70 (m, 2H), δ 3.83 (q, 6H, J=6.0 Hz), δ 7.00 (s, 1H), δ 7.40 (s, 1H). ¹³C NMR (100 MHz, DMSO-D6): δ 166, 137, 134, 128, 58, 43, 23, 18, and 7.6.

### Example 2—Preparation and Characterization of GSH-Responsive Silica Nanoparticles (SNPs)

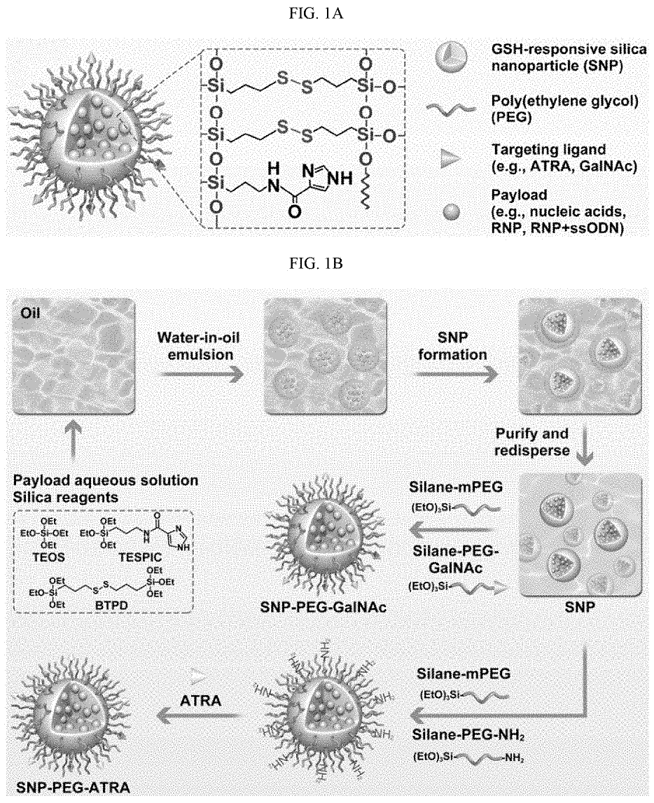

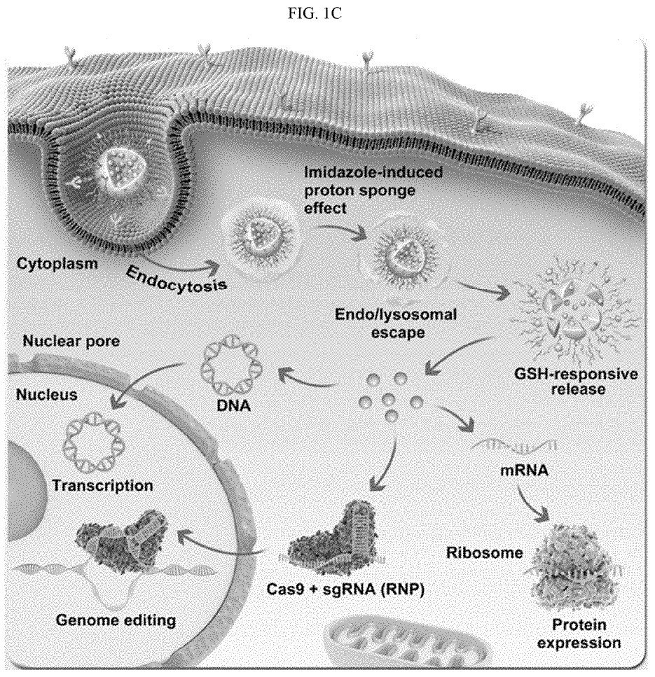

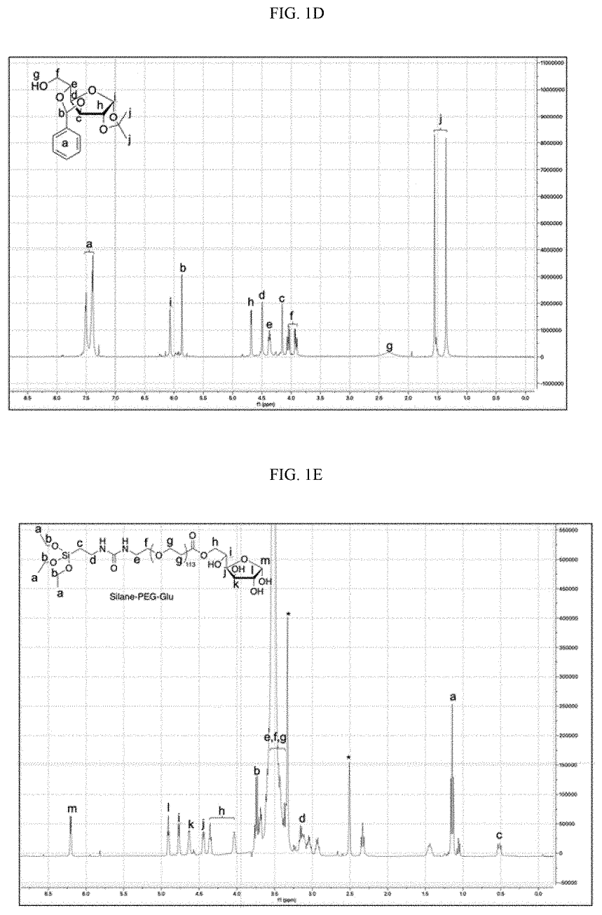

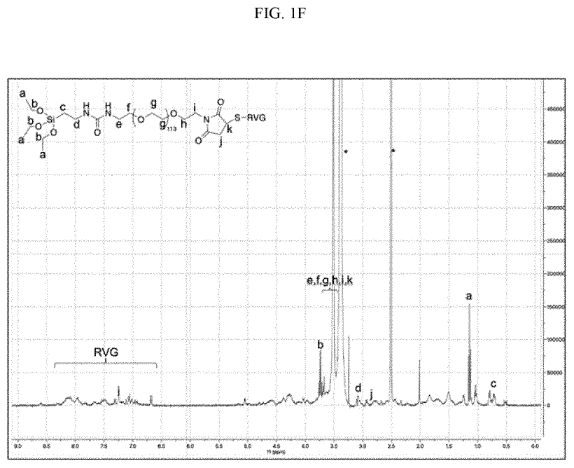

FIG. 1B depicts schematically how an illustrative embodiment of SNPs of the present technology (FIG. 1A) were synthesized by a water-in-oil emulsion method.

Preparation of SNP crosslinked silica network. Method A: Triton X-100 (1.8 mL) and hexanol (1.8 mL) were dissolved in cyclohexane (7.4 mL) to form the oil phase. Separately, 30 μL of a 5 mg/mL aqueous solution of desired biomolecule(s) (referred to as “the payload”, e.g., DNA, mRNA, RNP or RNP+ssODN) were mixed with TEOS (3.1 μL, 14 μmol), BTPD (6 μL, 13 μmol) and TESPIC (1 mg, 3 μmol for imidazole incorporation with 10% molar ratio, or 2 mg for 20% molar ratio). After shaking, this mixture was added to 1.1 mL of the oil phase, and then the water-in-oil microemulsion was formed by vortex for 1 min. Under stirring (1500 rpm), a 5 μL aliquot of 30% aqueous ammonia solution was added and the water-in-oil microemulsion was kept stirring at room temperature for 4 h to obtain unmodified SNPs with negative surface charge. To prepare positively charged SNPs (SNP-NH₂), the as-prepared SNP was modified with amine groups by the addition of APTES to the microemulsion, and the mixture was stirred vigorously for another 4 h at room temperature. To purify SNP or SNP-NH₂, 1.5 mL of acetone was added in the microemulsion in order to precipitate the SNPs, and the precipitates were recovered by centrifugation and washed twice with ethanol and three times with water. The purified SNP or SNP-NH₂ were finally collected by centrifugation.

Method B: The oil phase was prepared by mixing Triton X-100 (1.77 mL) with hexanol (1.8 mL) and cyclohexane (7.5 mL). An aliquot of aqueous solution (30 μL) containing the desired payload (e.g., DNA, mRNA, RNP or RNP+ssODN, 2 mg/mL) were mixed with the desired silica reagents (4 μL) (as shown in Table 1), BTPD (6 μL) and TESPIC (1 mg, 3 μmol). This mixture was homogenized by pipetting and then added to the oil phase (1.2 mL) The water-in-oil microemulsion was formed by vortex for one min. Under vigorous stirring (1,500 rpm), an aliquot of 30% aqueous ammonia solution (4 μL) was added and the water-in-oil microemulsion was stirred at 4° C. for 12 h to obtain unmodified SNPs. Acetone (1.5 mL) was added in the microemulsion to precipitate the SNPs, and the precipitate was recovered by centrifugation and was subsequently washed twice with ethanol and three times with water. The purified SNPs were finally collected by centrifugation. SNP1 prepared using Method B has the same formulation (structure) as SNP prepared using Method A. For in vivo studies (Example 11), four types of SNPs were conjugated glucose and RVG with a feed molar ratio of mPEG-silane:Glu-PEG-silane:RVG-PEG-silane=8:1:1.

TABLE 1

| Name | Silica reagent | Size (D [symbol] nm [symbol] ± 3D) | Zeta potential (mV₁ ± SD) |
|---|---|---|---|
| SNP1 | 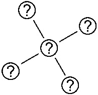 | 44 ± 3 | 0.8 ± 1.4 |
| SNP2 | 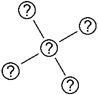 | 36 ± 1 | 1.9 ± 0.3 |
| SNP3 | 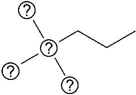 | 55 ± 7 | −3.0 ± 1.4 |
| SNP4 | 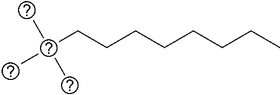 | 56 ± 7 | −3.8 ± 0.8 |
| SNP5 | 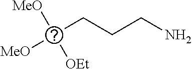 [SMILES: CCOC(CCCN)(OC)OC] | 48 ± 3 | 4.2 ± 0.4 |
| SNP6 | 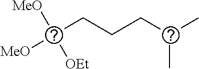 | 46 ± 3 | 4.6 ± 0.6 |
| SNP7 | 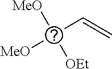 | [symbol] 1 ± 4 | 1.7 ± 1.8 |
| SNP8 | 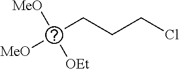 [SMILES: CCOC(CCCCl)(OC)OC] | 64 ± 1 | 3.5 ± 0.4 |

[symbol]

indicates data missing or illegible when filed

Preparation of PEGylated SNP (SNP-PEG). The as-prepared, unmodified SNP of Method A (2 mg) was re-dispersed in 2 mL water. An aliquot of mPEG-silane (for neutral surface charge, 200 μg) was added to the above mixture. The pH of the solution was adjusted to 8 using 0.1 M NaOH solution. The solution was stirred at room temperature for 4 h. The resulting SNP-PEG was purified by washing with water for three times and collected by centrifugation. For stability tests, mRNA-encapsulated SNP-PEG were redispersed in DI water with SNP concentration of 1 mg/ml and stored at different temperatures (i.e., 4° C., −20° C. and −80° C.); RNP encapsulated SNP-PEG were redispersed in RNP storage buffer (20 mM HEPES-NaOH pH 7.5, 150 mM NaCl, 10% glycerol), flash frozen in liquid nitrogen, and stored at −80° C.

Each as-prepared, unmodified SNP of Method B was re-dispersed in DI water (3 mL). For surface modification, mPEG-silane, or a mixture mPEG-silane+ silane-PEG-targeting ligand with different molar ratio was added to the above-mentioned SNP suspension. The total amount of PEG is 10 wt % of SNPs. The pH of the suspension was adjusted to 8.0 using 30% aqueous ammonia solution. The mixture was stirred at room temperature for 4 h. The resulting SNPs were purified by washing with DI water for three times and concentrated by Amicon® Ultra Centrifugal Filters (Millipore Sigma, USA).

Preparation of GalNAc-Conjugated SNP (SNP-PEG-GalNAc). GalNAc is known for its ability to bind with higher selectivity to the asialoglycoprotein receptors (ASGPRs) overexpressed on hepatocytes. To provide enhanced liver targeting capability to the SNP, the ligand, GalNAc was conjugated to the distal ends of the surface PEG. The as-prepared, unmodified SNP (2 mg) was re-dispersed in 2 mL water. An aliquot of GalNAc-PEG-silane (80 μg)+mPEG-silane (120 μg) (for SNP-PEG-GalNAc) was added to the above mixture. The pH of the solution was adjusted to 8 using 0.1 M NaOH solution. The solution was stirred at room temperature for 4 h. The resulting SNP-PEG-GalNAc was purified by washing with water for three times and collected by centrifugation.

Preparation of ATRA conjugated SNPs (SNP-PEG-ATRA). To provide retinal pigmented epithelium (RPE)-targeting capability to the SNP, the ligand, all-trams retinoic acid (ATRA) was conjugated to the distal ends of the surface PEG through amidation. The as-prepared, unmodified SNP (2 mg) was re-dispersed in 2 mL water. An aliquot of NH₂-PEG-silane (40 μg)+mPEG-silane (160 μg) was added to the above mixture. The pH of the solution was adjusted to 8 using 0.1 M NaOH solution. The solution was stirred at room temperature for 4 h. The resulting SNP-PEG-NH₂ was purified by washing with water for three times and collected by centrifugation. SNP-PEG-ATRA was synthesized via EDC/NHS catalyzed amidation. Briefly, payload-encapsulated SNP-PEG-NH₂ (1 mg) was re-dispersed in 0.5 mL DI water. EDC (15 μg), NHS (9 μg) and a DMSO solution of ATRA (12 μg in 10 μL DMSO) were added to the above solution. The solution was stirred at room temperature for 6 h, and then the resulting SNP-PEG-ATRA was washed with water three times and collected by centrifugation.

Preparation of TAT conjugated SNPs (SNP-PEG-TAT). SNP-PEG-TAT was synthesized via maleimide-thiol Michael addition. Payload-encapsulated SNP-PEG-Mal (1 mg) was re-dispersed in 1 mL DI water. An aqueous solution of TAT (120 μg in 12 μL DI water) and 0.5 M TECP aqueous solution (10 μL) were added to the above solution. The solution was stirred at room temperature for 6 h in nitrogen atmosphere, and then the resulting SNP-PEG-TAT was washed by water three times and collected by centrifugation.

### Synthesis of Silane-PEG-Glucose (Glu)

The synthesis scheme of the glucose conjugated PEG-silane is shown in Scheme A below; it is a three-step reaction.

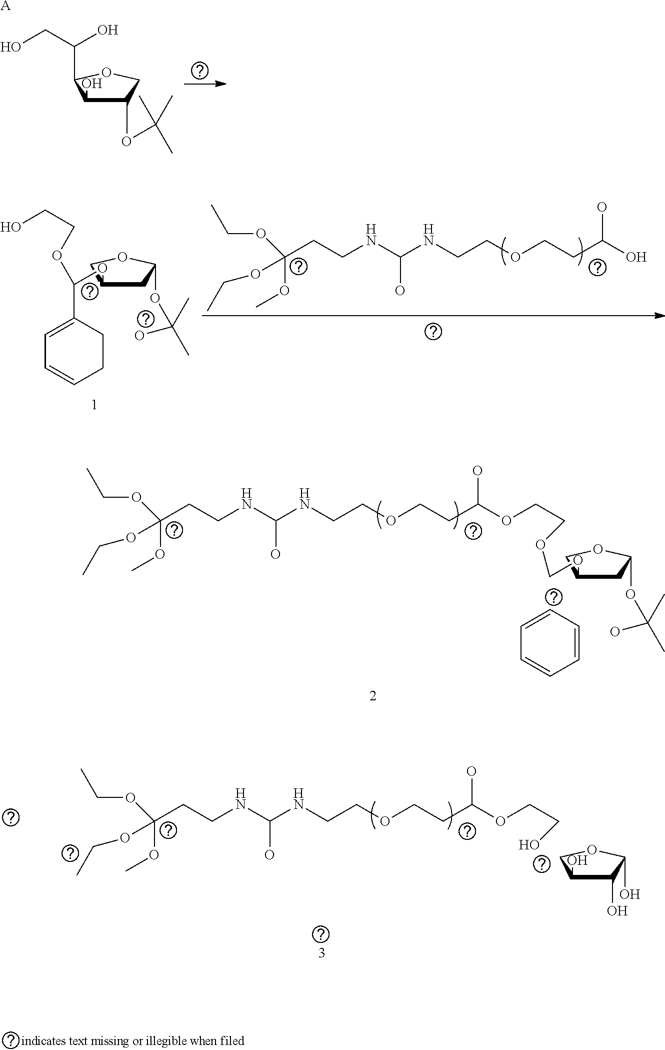

### Synthesis of 3,5-O-benzylidene-1,2-O-isopropylidene-α-D-glucofuranoside (BIG, Intermediate Reactant 1)

The intermediate reactant, 3,5-O-benzylidene-1,2-O-isopropylidene-α-D-glucofuranoside (1) was synthesized as reported previously (Y. Anraku, H. Kuwahara, Y. Fukusato, A. Mizoguchi, T. Ishii, K. Nitta, Y. Matsumoto, K. Toh, K. Miyata, S. Uchida, Glycemic control boosts glucosylated nanocarrier crossing the BBB into the brain, Nature Comm., 8 (2017) 1-9). Briefly, 3,5-O-benzylidene-1,2-O-isopropylidene-α-D-glucofuranoside (5 g, 23 mmol) and anhydrous zinc chloride (6.8 g, 50 mmol) were mixed in benzaldehyde (20 mL, 198 mmol) at room temperature under vigorous stirring overnight. The mixture was then dissolved in ethyl acetate (50 mL) and washed three time with DI water (50 mL). The organic phase was collected, dried over anhydrous sodium sulfate, and the organic solvent was removed by a rotary evaporator. The crude product was recrystallized from hexane to yield the final product (1) as a white solid. The ¹H NMR (CDCl₃, 400 MHz) spectrum (FIG. 1D) of the product was consistent with that expected.

### Synthesis of Silane-PEG-BIG (2)

BIG was conjugated to silane-PEG-COOH via an esterification reaction. BIG (92.4 mg, 0.3 mmol) and silane-PEG-COOH (1 g, 0.2 mmol) were dissolved in anhydrous DMF (10 mL), followed by the addition of EDC (62 mg, 0.4 mmol) and HOBt (54 mg, 0.4 mmol). The mixture was stirred at room temperature overnight and precipitated with diethyl ether (100 mL) to obtain the intermediate product silane-PEG-BIG without further purification.

### Synthesis of Silane-PEG-Glu (3)

Silane-PEG-BIG (150 mg) was dissolved in TFA/CH₂Cl₂ (1:1, v/v, 5 mL) and stirred for 1 h at room temperature. The solvent was then removed by a rotary evaporator. The residual was dissolved in anhydrous DMF (3 mL) and precipitated with diethyl ether (30 mL). The precipitate was washed twice with diethyl ether, and dried under vacuum to obtain the product silane-PEG-Glu without further purification. The ¹H NMR (DMSO-D6, 400 MHz) spectrum (FIG. 1E) of the product (3) was consistent with that expected.

### Synthesis of Silane-PEG-RVG

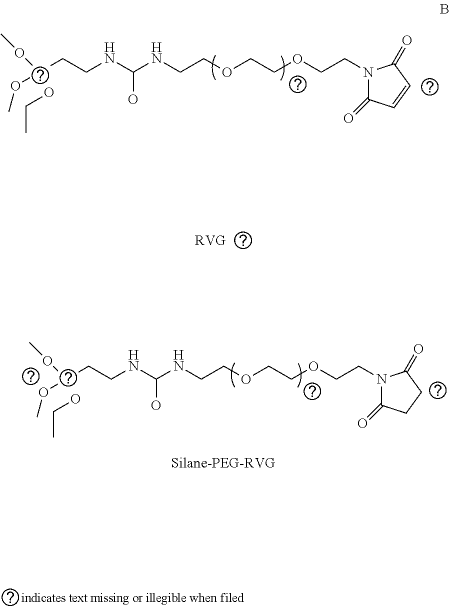

Silane-PEG-RVG was synthesized by a maleimide-thiol Michael addition reaction. RVG peptide (340 mg, 0.1 mmol) was mixed with silane-PEG-Mal (500 mg, 0.1 mmol) and dissolved in anhydrous DMF (10 mL). TCEP (57.4 mg, 0.2 mmol) was added to the mixture, and the reaction was carried out at room temperature for 16 h in a nitrogen atmosphere. The product, silane-PEG-RVG, was purified by precipitation with diethyl ether (50 mL) for 3 times and dried under vacuum. The product was directly used for experiments without further purification. The ¹H NMR (DMSO-D6, 400 MHz) spectrum (FIG. 1F) of the product was consistent with that expected.

### Preparation of the GSH-Responsive Silica Nanoparticles (SNPs)

SNPs were synthesized by a water-in-oil microemulsion method using a previously reported method. (Y. Wang, P. K. Shahi, X. Wang, R. Xie, Y. Zhao, M. Wu, S. Roge, B. R. Pattnaik, S. Gong, In vivo targeted delivery of nucleic acids and CRISPR genome editors enabled by GSH-responsive silica nanoparticles, Journal of Controlled Release, (2021)); E. A. Prasetyanto, A. Bertucci, D. Septiadi, R. Corradini, P. Castro-Hartmann, L. De Cola, Breakable hybrid organosilica nanocapsules for protein delivery, Angewandte Chemie International Edition, 55 (2016) 3323-3327.) The oil phase was prepared by mixing Triton X-100 (1.77 mL) with hexanol (1.8 mL) and cyclohexane (7.5 mL). An aliquot of aqueous solution (30 μL) containing the desired payload (e.g., DNA, mRNA, RNP or RNP+ssODN, 2 mg/mL) were mixed with the desired silica reagents (4 μL) (as shown in Table 1), BTPD (6 μL) and TESPIC (1 mg, 3 μmol). This mixture was homogenized by pipetting and then added to the oil phase (1.2 mL) The water-in-oil microemulsion was formed by vortex for one min. Under vigorous stirring (1,500 rpm), an aliquot of 30% aqueous ammonia solution (4 μL) was added and the water-in-oil microemulsion was stirred at 4° C. for 12 h to obtain unmodified SNPs. Acetone (1.5 mL) was added in the microemulsion to precipitate the SNPs, and the precipitate was recovered by centrifugation and was subsequently washed twice with ethanol and three times with water. The purified SNPs were finally collected by centrifugation.

The as-prepared, unmodified SNP was re-dispersed in DI water (3 mL). For Surface modification, mPEG-silane, or a mixture mPEG-silane+ silane-PEG-targeting ligand with different molar ratio was added to the above-mentioned SNP suspension. The total amount of PEG is 10 wt % of SNPs. The pH of the suspension was adjusted to 8.0 using 30% aqueous ammonia solution. The mixture was stirred at room temperature for 4 h. The resulting SNPs were purified by washing with DI water for three times and concentrated by Amicon® Ultra Centrifugal Filters (Millipore Sigma, USA).

### Example 3—SNP Characterization

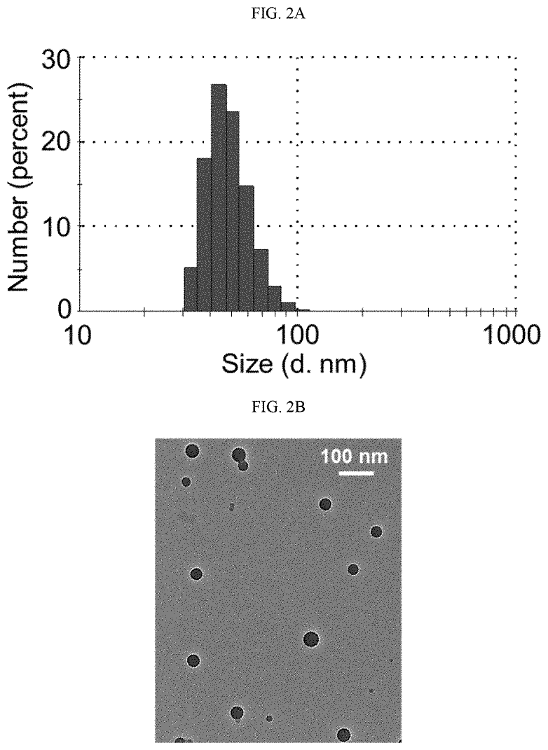

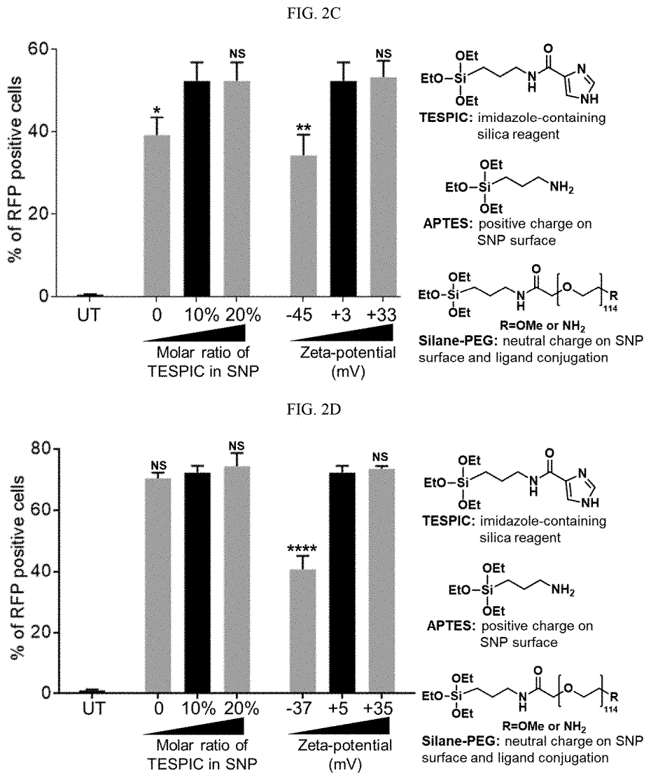

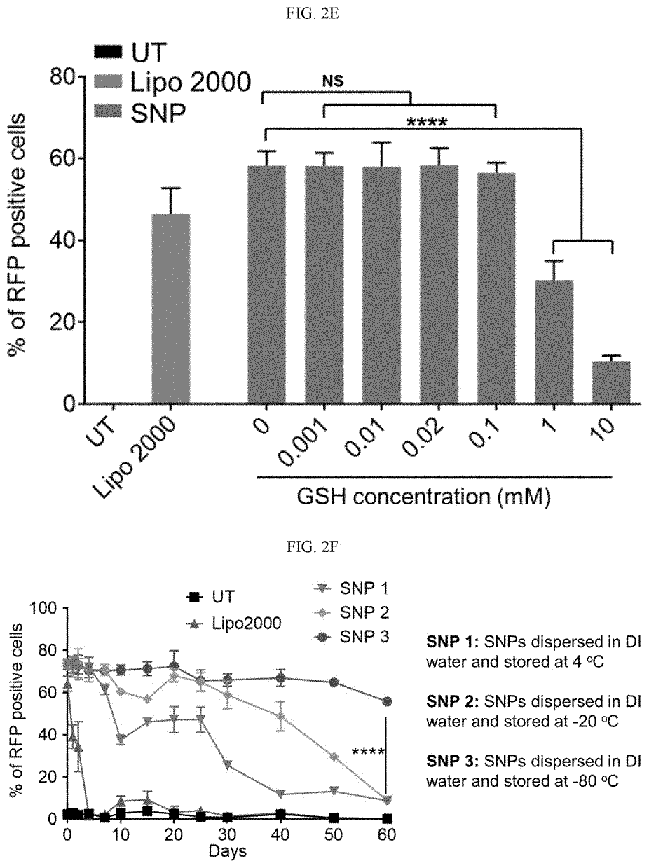

A variety of biomacromolecules were encapsulated into SNPs, including plasmid DNA, mRNA, RNP and the mixture of RNP and donor oligonucleotide for gene correction (i.e., RNP+ssODN). The hydrodynamic diameter, zeta-potential, loading content and loading efficiency of PEGylated SNPs with different payloads are summarized in Tables 1 (3-arm and 4-arm SNPs) and 2 (4-arm SNPs). The morphology of the DNA-loaded SNP-PEG was characterized by transmission electron microscopy (TEM, Tecnai 12, Thermo Fisher, USA). FIG. 2A shows a TEM image of the PEGylated SNPs with spherical structure and an average size of 35 nm. The hydrodynamic diameter of DNA-loaded 4-arm SNP-PEG was 45 nm, as measured by dynamic light scattering (DLS) (FIG. 2B). The zeta-potential of DNA-loaded 4-arm SNP-PEG was 6.4 mV, indicating a nearly neutral surface charge after PEGylation. The size and zeta-potential of 4-arm SNP-PEG was found independent of the payload. As shown in Table 1, SNPs formed by different silica reagents exhibited similar sizes and nearly neutral surface charges, indicating that the components of SNP did not significantly affect their sizes and levels of PEGylation.

TABLE 2
Summary of SNP-PEG size, zeta-potential, loading
content and loading efficiency of different payloads.

| | Hydro- | Zeta- | Loading | Loading |
|---|---|---|---|---|
| | dynamic | potential | content | efficiency |
| Payload | diameter (nm) | (mV) | (wt %) | (%) |
| DNA | 45 | 6.4 | 9.0 | 90 |
| mRNA | 46 | 3.0 | 9.2 | 91 |
| RNP | 52 | 6.5 | 9.1 | 90 |
| RNP + ssODN | 49 | 5.9 | 9.4 | 93 |

For hydrophilic biomolecules, the loading contents varied between 9.0-9.4 wt %, with an overall high loading efficiency of ≥90%. In particular, there was no significant difference in loading content and loading efficiency between payloads, indicating that the SNP is a versatile nanoplatform for nucleic and protein encapsulation.

### Example 4—Determination of Transfection Efficiencies

The SNP formulation was optimized in HEK 293 cells to achieve high transfection efficiencies, using DNA and mRNA as payloads, separately. The weakly basic group, imidazole, was expected to enhance the endo/lysosomal escape capability of the SNP-PEG (FIG. 1C). Therefore, the ratio of imidazole in the SNPs can be a factor for efficient nucleic acid delivery. The optimal ratio of imidazole-containing reactant TESPIC in the SNP was investigated by fixing the feed molar ratio of TEOS and BTPD. As shown in FIG. 2C, SNP-PEG with 10 mol % imidazole-containing TESPIC exhibited higher DNA transfection efficiency (1.3-fold) than the one without TESPIC, while further increasing the TESPIC molar ratio does not lead to higher DNA transfection efficiency. The TESPIC ratio in mRNA-encapsulated SNP-PEG was investigated, but mRNA delivery efficiency was independent of the TESPIC ratio.

To investigate the influence of SNP surface charges on nucleic acid delivery efficiencies, we prepared DNA- and mRNA-encapsulated SNPs with different surface charges (FIGS. 2C and 2D). The as-prepared, unmodified SNPs had a strong negative zeta-potential; positively charged SNPs (i.e., SNP-NH₂) and neutral PEGylated SNPs (SNP-PEG) were prepared by APTES and mPEG-silane conjugation, respectively. As shown in FIG. 2C, SNP-NH₂ exhibited a 1.6-fold higher DNA transfection efficiency and a 1.8-fold higher mRNA transfection efficiency than negatively charged SNP. SNP-PEG with a neutral surface charge exhibited similar DNA and mRNA transfection efficiencies, indicating that moderate surface PEGylation does not affect SNP uptake by cells.

Disulfide bonds were integrated into the SNP to facilitate payload release in the cytosol with a high GSH concentration (2-10 mM). To ensure extracellular GSH (0.001-0.02 mM) did not cause stability concerns or induce premature cargo release, the GSH-responsive behavior of SNP was investigated. DNA encapsulated SNP-PEG was incubated with HEK 293 cells in culture media containing intentionally added GSH with a GSH concentration ranging from 0-10 mM. As shown in FIG. 2E, the DNA transfection efficiency was not affected at GSH concentrations equal to or lower than 0.1 mM, suggesting that the SNP is stable in the extracellular space. However, a significant decrease in the DNA transfection efficiency was observed at a GSH concentration of 1 mM or higher, suggesting that the SNP are not stable at high GSH concentrations, therefore, they can effectively break down in the cytosol to release the payload.

The stability of mRNA-loaded SNP-PEG after long-term storage was also studied. The mRNA transfection efficiency of SNP-PEG was intact after 60-day storage at −80° C., or 25 days at 4° C. or −20° C. (FIG. 2F), indicating SNP-PEG is desirable for future biomedical applications.

### Example 5—Intracellular Trafficking of SNPs

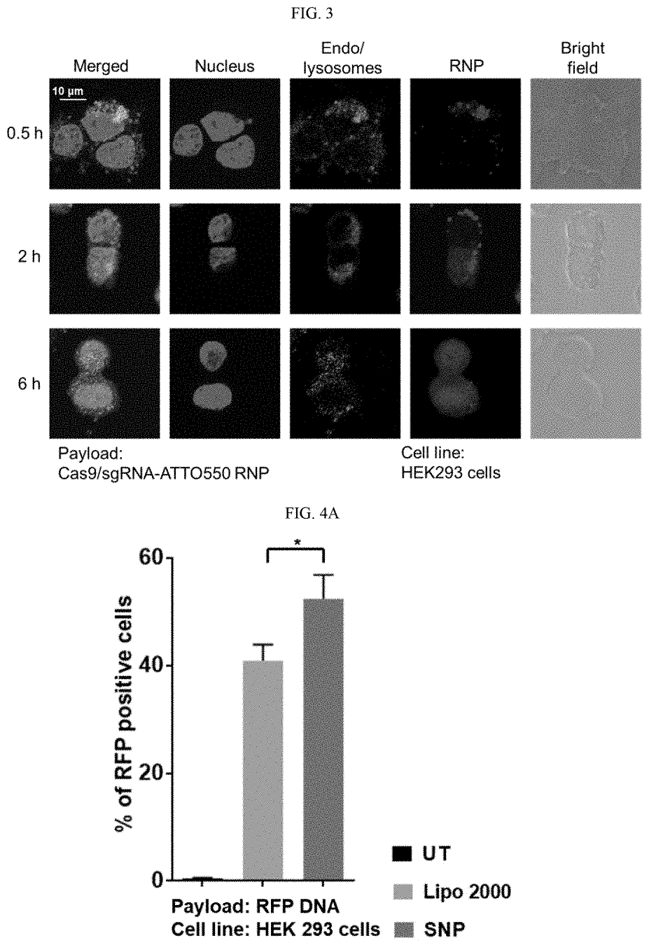

The intracellular trafficking of RNP-encapsulated SNP-PEG was studied by confocal laser scanning microscopy (CLSM) in HEK 293 cells (FIG. 3). Payload RNP was prepared by mixing the NLS-tagged Cas9 and ATTO-550-tagged guide RNA. After incubating RNP-loaded SNP-PEG with cells for 0.5 hours, RNP was mainly co-localized with endo/lysosomes, indicating the internalization of SNP-PEG via endocytosis. Endo/lysosomal escape of the SNP-PEG assisted by imidazole was observed 2 h post-treatment, indicated by the decrease of co-localized RNP and endo/lysosome signals. The RNP signal showed considerable overlap with the nucleus and further decreased co-localization with endo/lysosomes 6 h post-treatment, indicating the successful nuclear transportation of RNP induced by the NLS tags on the RNP.

### Example 6—Comparison of SNP Biomolecule Delivery Efficiency to Commercial Products

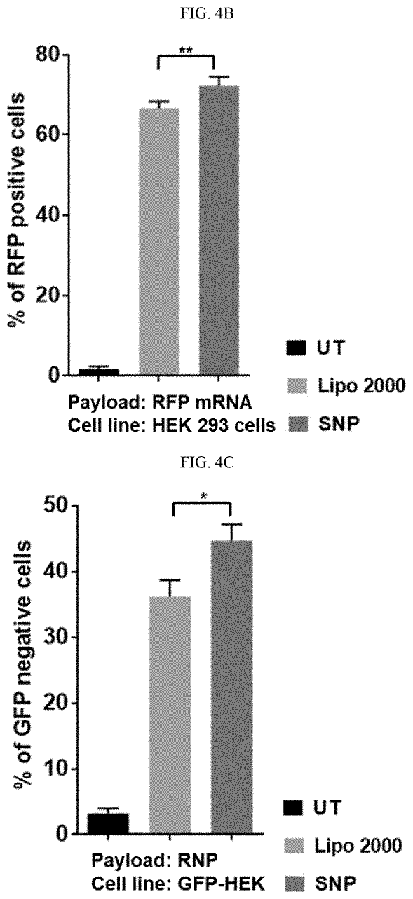

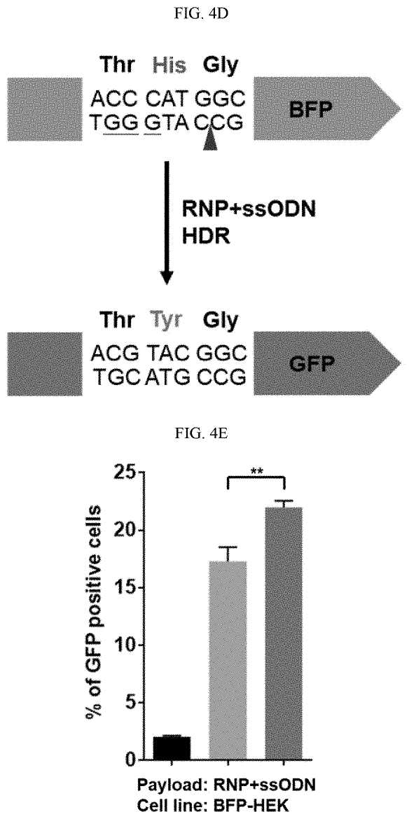

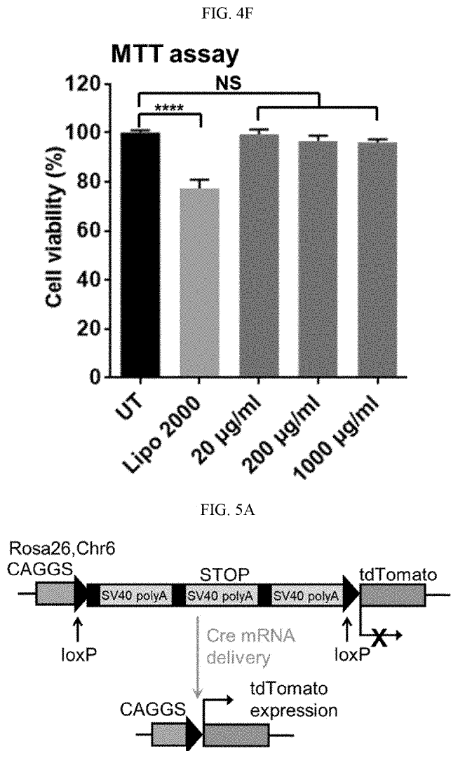

To investigate the versatility of SNPs for biomolecule delivery, HEK 293 cells were used for nucleic acid delivery/genome editing efficiency studies, and flow cytometry was used to quantify the delivery efficiency. The DNA and mRNA transfection efficiency by SNP-PEG were tested in HEK293 cells (FIGS. 4A and 4B). SNP-PEG exhibited statistically higher DNA and mRNA transfection efficiency (1.3-fold and 1.1-fold, respectively) than the commercially available transfection reagent Lipofectamine 2000 (Lipo 2000), indicating the superior nucleic acid delivery capability of SNPs.

The CRISPR-Cas9 RNP is a fast, efficient and accurate genome editing machinery. Cas9 as a nuclease can cause double-stranded DNA break in a specific genomic locus under the guidance of gRNA, achieving gene deletion by the nonhomologous end joining (NHEJ) DNA repair pathway. Moreover, with a donor DNA template (e.g., single-stranded oligonucleotide DNA (ssODN)) delivered together with RNP, gene correction or insertion can be achieved through the homology-directed repair (HDR) pathway. The genome-editing efficiency of SNP-PEG was investigated by delivering the RNP targeting the GFP gene in a transgenic GFP-expressing HEK 293 cell line. As shown in FIG. 4C, RNP-encapsulated SNP-PEG exhibited a significantly higher gene-knockout efficiency (1.3-fold) than Lipo 2000. To investigate gene correction capability of SNPs, a BFP-expressing HEK 293 cell line was used. Precise gene editing by HDR will lead to the replacement of three nucleotides in the genome, thereby altering one histidine to tyrosine (FIG. 4D), which leads to the BFP to GFP conversion. RNP targeting the BFP gene and a donor ssODN were co-encapsulated into SNP-PEG. The genome-editing efficiency was evaluated by the percentage of GFP-positive cells. As shown in FIG. 4E, SNPs exhibited a statistically higher (1.1-fold) gene-correction efficiency than Lipo 2000. These results demonstrate the capability of SNPs as an efficient nanoplatform for genome editing machinery delivery.

The biocompatibility of SNPs was evaluated. HEK 293 cells were treated with DNA-encapsulated SNP-PEG at different SNP-PEG concentrations, and the cell viability was studied by an MTT assay. As shown in FIG. 4F, SNP-PEG did not induce significant cytotoxicity in HEK293 cells with concentrations up to 1000 μg/mL, 45-times higher than the working concentration used for our delivery efficiency studies. However, at the working DNA concentration, DNA-complexed Lipo 2000 showed only 77% cell viability, indicating a significantly higher cytotoxicity than SNP-PEG. These results show that the SNPs are desirable nanoplatform for efficient delivery of various biomacromolecules.

### Example 7—In Vivo SNP Biomolecule Delivery Efficiency

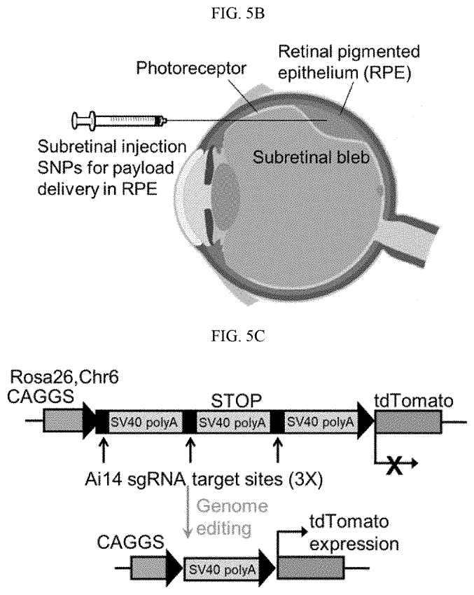

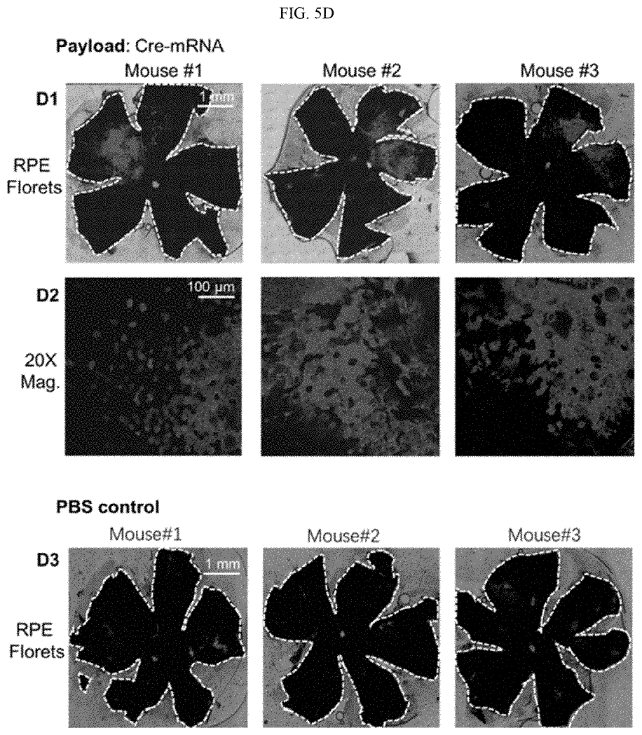

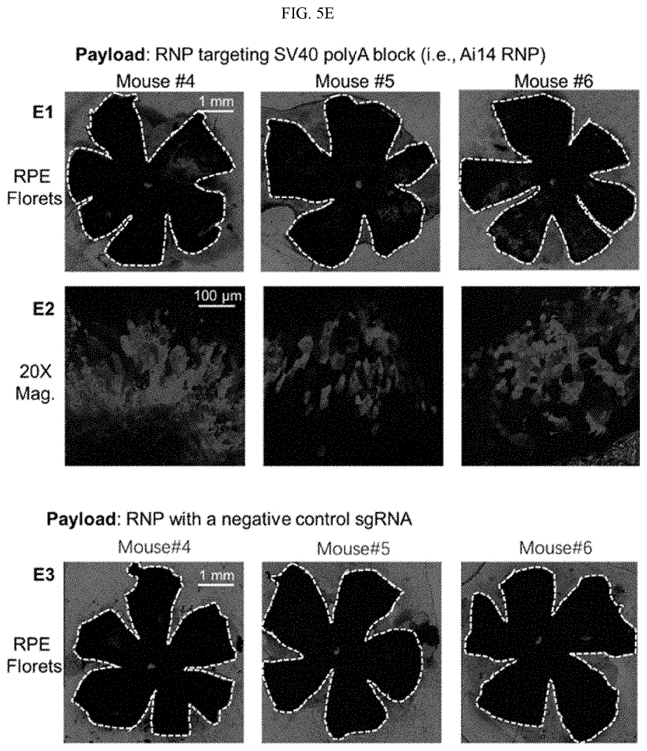

Nucleic acid delivery/genome editing efficiency of SNPs were further investigated in transgenic Ai14 mice (FIG. 5). The Ai14 mouse genome contains a CAGGS promoter and a LoxP-flanked stop cassette with three repeats of the SV40 polyA sequence, preventing the expression of the downstream tdTomato fluorescent protein gene. The gain-of-function fluorescence can be achieved by: 1) Cre-Lox combination via the delivery of Cre recombinase or Cre-encoding DNA/mRNA (FIG. 5A), or 2) excision of 2 of the SV40 polyA blocks by Cas9 RNP (FIG. 5C). The tdTomato fluorescence signal in cells edited by Cre-encoding DNA/mRNA (FIG. 5A) provides a quantitative readout of nucleic acid delivery/genome editing in Ai14 mice. However, the tdTomato fluorescence signal in cells edited by Cas9 RNP (FIG. 5C) greatly under reports the editing efficiency (B. T. Staahl, M. Benekareddy, C. Coulon-Bainier, A. A. Banfal, S. N. Floor, J. K. Sabo, C. Urnes, G. A. Munares, A. Ghosh, J. A. Doudna, Efficient genome editing in the mouse brain by local delivery of engineered Cas9 ribonucleoprotein complexes, Nature Biotechnology, 35 (2017) 431-434).

To study mRNA delivery efficiency by SNPs, eyes of Ai14 mice were subretinally injected with a Cre-mRNA-encapsulated SNP-PEG-ATRA (FIG. 5B); subretinal injection of PBS was used as a control. Four days post injection, RPE tissues were separated from the eye and flat-mounted, tdTomato expression in the flattened RPE tissue (i.e., RPE floret) was studied by confocal laser scanning microscopy. As shown in FIG. 5D, strong tdTomato fluorescence was visualized in the RPE florets with SNP-PEG-ATRA injection, indicating efficient delivery of Cre-mRNA by SNPs. Moreover, the genome editing efficiency of SNPs was studied by subretinal injection of Cas9 RNP encapsulated SNPs. Mice were subretinally injected with a SNP-PEG-ATRA encapsulating the RNP targeting the SV40 polyA block (i.e., Ai14 RNP), or a SNP-PEG-ATRA encapsulating the RNP with negative control sgRNA (i.e., negative control). The tdTomato expression was evaluated 14 days post-injection. As shown in FIG. 5E, Ai14 RNP-loaded SNPs induced robust tdTomato expression in the RPE, the ratio of tdTomato positive area to total RPE floret was calculated as 4.5%. No tdTomato signal was found in eyes injected with negative control SNPs. These results suggest that SNP is a reliable nanoplatform for in vivo biomacromolecule delivery.

### Example 8—Use of CPP-Tagged SNP to Induce SNP Uptake into Cells

The wild-type human induced pluripotent stem cells (hiPSCs, ACS-1011, ATCC, USA) were cultured on mouse embryonic fibroblasts (MEFs) in iPS cell medium (Dulbecco's modified Eagle's medium (DMEM):F12 (1:1), 20% KnockOut Serum, 1% minimal essential medium (MEM), non-essential amino acids, 1% GlutaMAX, β-mercaptoethanol, and 20 ng/mL fibroblast growth factor 2 (FGF-2)). The hiPSCs were differentiated to retinal pigment epithelium (RPE) using known protocols (Shahi P K, et al. “Gene augmentation and readthrough rescue channelopathy in an iPSC-RPE model of congenital blindness” Am. J. Hum. Genet. 2019, 104(2):310-8; Meyer J S, Shearer R L, Capowski E E, Wright L S, Wallace K A, McMillan E L, Zhang S-C, Gamm D M. Modeling early retinal development with human embryonic and induced pluripotent stem cells. Proc. Natl. Acad. Sci. U.S.A. 2009, 106(39):16698-703). In brief, hiPSCs were lifted enzymatically and grown as embryoid bodies (EBs) in iPS cell medium without FGF-2. The medium was gradually changed to neural induction medium (NIM; DMEM:F12; 1% N₂ supplement, 1% MEM non-essential amino acids, 1% L-glutamine and 2 μg/mL heparin) by day 4. At day 7, free-floating EBs were plated on laminin-coated culture plates so that the cell aggregates were allowed to adhere to the plates. At day 16, the aggregates were removed, and the medium was switched to retinal differentiation medium (DMEM/F12 [3:1], 2% B27 supplement (without retinoic acid), and 1% Antibiotic-Antimycotic). Remaining adhered cells were allowed to continue differentiation for an additional 45 days. Monolayered hiPSC-RPE cells were purified by microdissection and passaging, as described earlier (Singh R, et al. “Functional analysis of serially expanded human iPS cell-derived RPE cultures” Invest. Ophth. Vis. Sci. 2013, 54(10):6767-78).

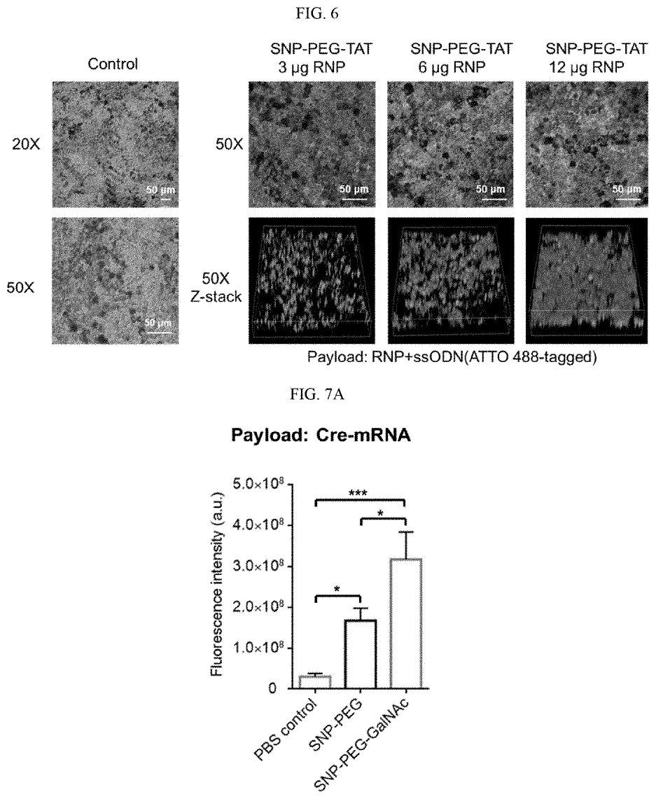

The delivery efficiency of SNP was tested in iPSC-RPE cells. hiPSC-RPE is a promising alternative to human RPE for genetic studies, it has been shown to display identical characteristics of mature human RPE. RNP with a donor ssODN (RNP+ssODN) with a 1:1 molar ratio was encapsulated into a cell penetrating peptide (i.e., TAT)-modified SNP, SNP-PEG-TAT. The donor sequence, ssODN, was tagged with a green fluorescence dye, ATTO-488. iPSC-RPE was treated with RNP+ssODN-loaded SNP-CPP at different dosages, and the cellular uptake of the payload was evaluated by CLSM. Four days post-treatment, significant cellular uptake in iPSC-RPE was observed, and the uptake efficiency was dose-dependent (FIG. 6). In addition, no alternation in RPE cell morphology and density was observed, indicating that the high-dosage SNP treatment and cellular uptake did not induce cytotoxicity in hiPSC-RPE cells.

### Example 9—Genome Editing in Liver Via Intravenous Injection

The nucleic acid and RNP delivery efficiency of intravenously injected SNP was also evaluated in vivo using Ai14 mice. Two types of SNPs were involved in this study: (1) SNP-PEG and (2) liver-targeting SNP-PEG-GalNAc. Liver was chosen as the target organ because it is an important target for therapeutics development. Nanoplatforms capable of safe and efficiency gene/gene editor delivery to liver can be powerful tools for the treatment of liver diseases (e.g., nonalcoholic fatty liver disease, liver cancer and hereditary tyrosinemia).

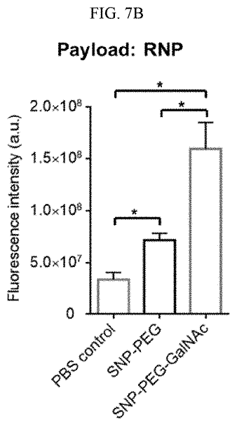

Cre-mRNA delivery was investigated with an mRNA dosage of 20 μg per mouse. Major organs were collected 3 days post injection, and the tdTomato fluorescence was analyzed by IVIS (photomicrographs not shown). Although tdTomato signal was mainly detected in the liver for both non-targeted and targeted SNPs, the SNP-PEG-GalNAc injected mice exhibited a stronger liver tdTomato signal than SNP-PEG (photomicrographs not shown). The homogenized liver tissue showed a 2-fold increase of tdTomato signal in the liver of SNP-PEG-GalNAc injected mice than the SNP-PEG group (FIG. 7A), indicating GalNAc conjugation on the SNP surface can further enhance liver targeting efficiency. To confirm the tdTomato expression, liver sections were immunofluorescence stained with anti-tdTomato antibody and then fluorescein-tagged secondary antibody. The immunostained liver sections were examined using confocal fluorescence microscopy. tdTomato-positive cells were found in liver tissue, while tdTomato positive cells were not detected in the PBS-injected mice (photomicrographs not shown), indicating that SNPs, with or without GalNac, can deliver mRNA into liver via systemic administration.

RNP delivery was investigated with RNP encapsulated SNP or SNP-PEG-GalNAc (100 μg RNP per mouse). Major organs were collected 7 days post-injection. Similar to Cre mRNA, tdTomato signal were mainly found in the liver (photomicrographs not shown), and SNP-PEG-GalNAc showed a 2-fold higher gene editing efficiency than SNP-PEG, as quantified by the fluorescence intensity of homogenized tissue (FIG. 7B). Immunofluorescence staining of sectioned liver showed strong tdTomato expression induced by RNP delivery (photomicrographs not shown).

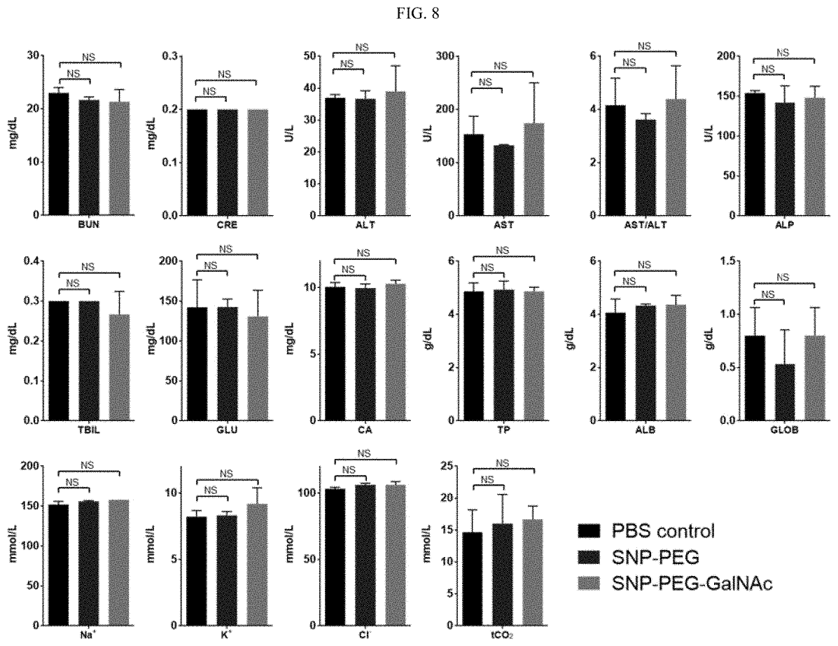

To evaluate the potential systemic toxicity of SNP, a blood biochemistry test was performed for all the injected mice (FIG. 8). The key elements of the blood biochemical profile (e.g., total CO₂, ALT, AST, BUN, etc.) showed no significant difference between SNP-injected groups and the PBS control group, indicating that the SNP possessed good biocompatibility. This proof-of-principle data indicates that intravenous administration of SNP can achieve gene delivery/gene editing in vivo. Furthermore, SNP conjugated with targeting moieties can further enhance the biomolecule delivery efficiency in targeted tissues/cells.

### Example 10—In Vitro Delivery Efficiency of 3-Arm SNPs for DNA, mRNA and RNP

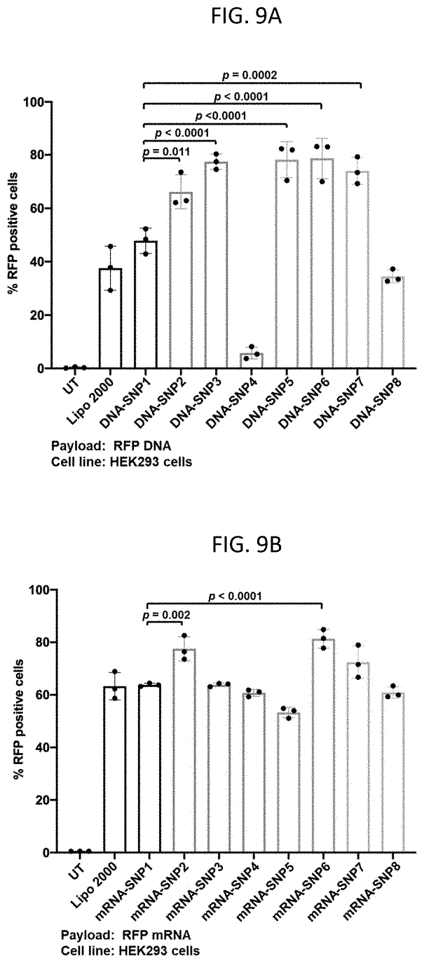

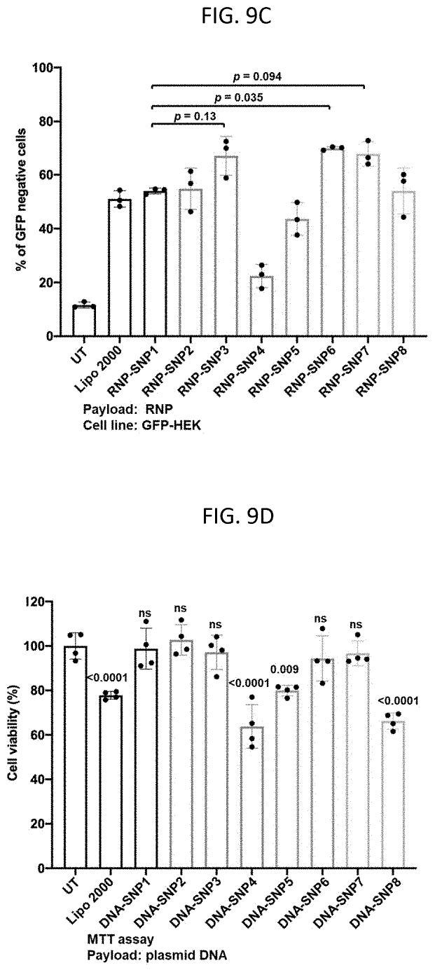

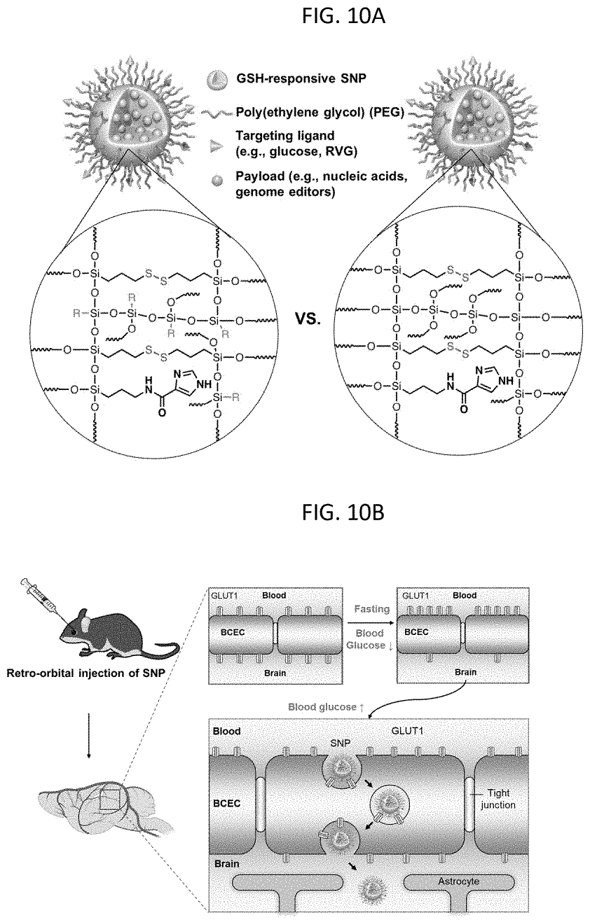

The delivery efficiency of several SNPs from Table 1 was investigated in vitro as described in the General Procedures and as in Example 6. Example 9 above shows that the SNP formed by TEOS (i.e., SNP1) can be used for nucleic acid/genome editor delivery. The delivery efficiency of different payloads (i.e., DNA, mRNA and RNP) by SNPs composed of silica reagents containing an inactive arm carrying different moieties (i.e., SNP2-SNP8 which are 3-arm SNPs) were compared with (4-arm) SNP1 (FIG. 10A schematically shows the differences between 3-arm and 4-arm structures in the SNPs). The SNPs exhibited distinct delivery performance for different payload types. For DNA delivery (FIG. 9A), a number of SNPs (i.e., DNA-SNPs) showed a higher transfection efficiency (up to two-fold) than the commercially available agent, Lipofectamine 2000 (Lipo 2000), including SNPs with short fatty chain inactive arms (i.e., DNA-SNP2 and DNA-SNP3), SNPs with charged inactive arms (i.e., DNA-SNP5 and DNA-SNP6), and an SNP with an unsaturated inactive arm (i.e., DNA-SNP7). For mRNA delivery (FIG. 9B), mRNA-SNP2, mRNA-SNP6 and mRNA-SNP7 showed an increased transfection efficiency in comparison with Lipo 2000 and mRNA-SNP1. The genome-editing efficiency of RNP-encapsulated SNPs (i.e., RNP-SNP) was determined by delivering an RNP targeting the GFP gene in a transgenic GFP-expressing HEK 293 cell line. As shown in FIG. 9C, three SNP formulations showed up to 1.6-fold higher gene editing efficiency than Lipo 2000 or RNP-SNP1, namely, the two SNPs with short fatty chain inactive arms (i.e., RNP-SNP2 and RNP-SNP3), and the SNP with tertiary amine inactive arm (i.e., RNP-SNP6). The MTT assay was performed to study the biocompatibility of DNA-SNPs (FIG. 9D). DNA-SNP1 and most of the SNP formulations (i.e., DNA-SNP2, DNA-SNP3, DNA-SNP6 and DNA-SNP7) with enhanced nucleic acid/gene editor delivery efficiencies exhibited negligible cytotoxicity.

### Example 11—In Vivo Delivery Efficiency of mRNA and RNP Using Brain-Targeted Intravenously Injected SNPs

The in vivo RNP and mRNA delivery efficiency of SNPs in Ai14 mice was determined as described in the General Procedures and Example 7. Ai14 mouse genome contains a CAGGS promoter and a LoxP-flanked stop cassette (i.e., three SV40 polyA sequences), which prevents the expression of the downstream tdTomato gene. Cre recombinase-encoding mRNA (i.e., Cre mRNA), or CRISPR RNP targeting the SV40 polyA sequence can remove the stop cassette and lead to tdTomato expression in the targeted tissue/cells, which can be used to detect the transfection/gene editing efficiency in vivo. However, the editing efficiency of RNP is significantly under-reported based on the tdTomato expression level using the Ai14 report mice, because tdTomato expression requires multiple RNP-mediated DNA breaks to generate excisions of at least two of three SV40 polyA sequences (B. T. Staahl, M. Benekareddy, C. Coulon-Bainier, A. A. Banfal, S. N. Floor, J. K. Sabo, C. Urnes, G. A. Munares, A. Ghosh, J. A. Doudna, Efficient genome editing in the mouse brain by local delivery of engineered Cas9 ribonucleoprotein complexes, Nature Biotechnology, 35 (2017) 431-434).

GLUT1 is expressed on the BCECs (FIG. 10B). Upon fasting (i.e., 16-24 h fasting), the density of GLUT1, glucose transporter 1, on the luminal side of the cell membrane is increased. When the blood glucose is rapidly restored, GLUT1 on the luminal side starts to migrate to the abluminal side of the BCEC membrane, presumably through a transcytosis process. Nanocarriers conjugated with glucose can cross the BBB utilizing this mechanism.

The effects of targeting ligands (types and amounts) on the ability of the SNPs to cross the BBB were first investigated using RNP-encapsulated SNP1 (i.e., RNP-SNP1) using Ai14 mice. RNP-SNP1 with different surface modifications (i.e., feed molar ratios of 10 mol % glucose+10 mol % RVG (RNP-SNP1-Glu+RVG), 10 mol % glucose (RNP-SNP1-10% Glu), 5 mol % glucose (RNP-SNP1-5% Glu) and SNP1 without targeting ligand (RNP-SNP1-no ligand)) were retro-orbitally injected into the Ai14 mice after 24 h fasting. Thirty minutes post SNP injection, the blood glucose was restored by intraperitoneal injection of glucose solution. Two weeks post SNP injection, mice were perfused with ice-cold PBS and major organs were collected for further processing.

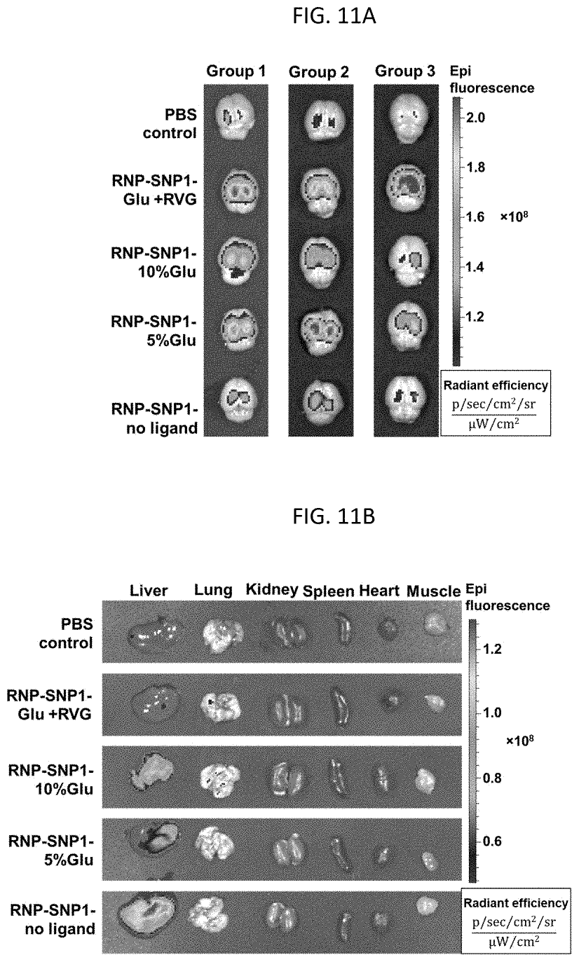

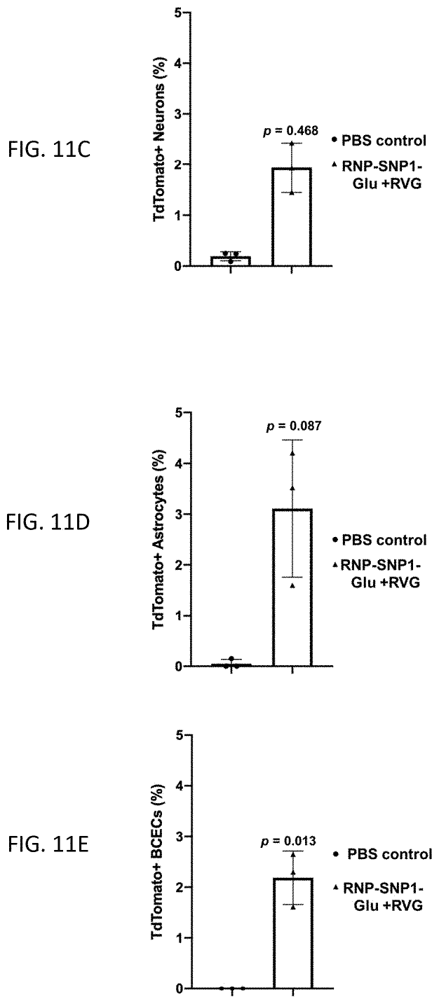

Ex vivo MS imaging showed RNP-SNP1 with a dual targeting ligand (i.e., RNP-SNP1-Glu+RVG, feed molar ratio of mPEG-silane:Glu-PEG-silane:RVG-PEG-silane=8:1:1) exhibited the highest tdTomato expression in the brain (FIG. 11A). RNP-SNP1 with only glucose modification showed moderate tdTomato signal (FIG. 11A), while RNP-SNP1 without targeting ligand and with glycemic control showed tdTomato expression in the liver but barely in the brain (FIG. 11B). Based on this study, we have determined that SNP conjugated with both glucose and RVG offer the best brain targeting capability and will be used for the following in vivo studies.

The editing efficiency of RNP-SNP1-Glu+RVG was further analyzed by fluorescence-activated cell sorting (FACS), a technique capable of identifying the types and levels of edited cells. Brains of SNP-administered mice as well as the brain of PBS control mice were collected, brain cells were isolated for further immunostaining using a previously reported protocol (F. J. Rubio, X. Li, Q.-R. Liu, R. Cimbro, B. T. Hope, Fluorescence activated cell sorting (FACS) and gene expression analysis of Fos-expressing neurons from fresh and frozen rat brain tissue, JoVE (Journal of Visualized Experiments), (2016) e54358; E. E. Crouch, F. Doetsch, FACS isolation of endothelial cells and pericytes from mouse brain microregions, Nature Protocols, 13 (2018) 738-751). Dissociated brain cells were fixed and immunofluorescence stained with cell markers, namely, NeuN (post-mitotic neuronal marker protein), GFAP (an astrocyte marker protein), and CD31 (also known as PECAM1, a BCEC marker protein), as well as anti-Tdtomato antibody to confirm the tdTomato expression. As shown in FIGS. 11 C-E, RNP-SNP1-Glu+RVG induced about 2% tdTomato expressing cells in neurons and BCECs, as well as 3% in astrocytes. However, as stated earlier, the genome editing efficiency of RNP is greatly underestimated by the tdTomato expression level. For the subsequent in vivo studies, we decided to use Cre mRNA as a payload because Ai14 reporter mice can accurately reflect the transfection efficiency of Cre mRNA.

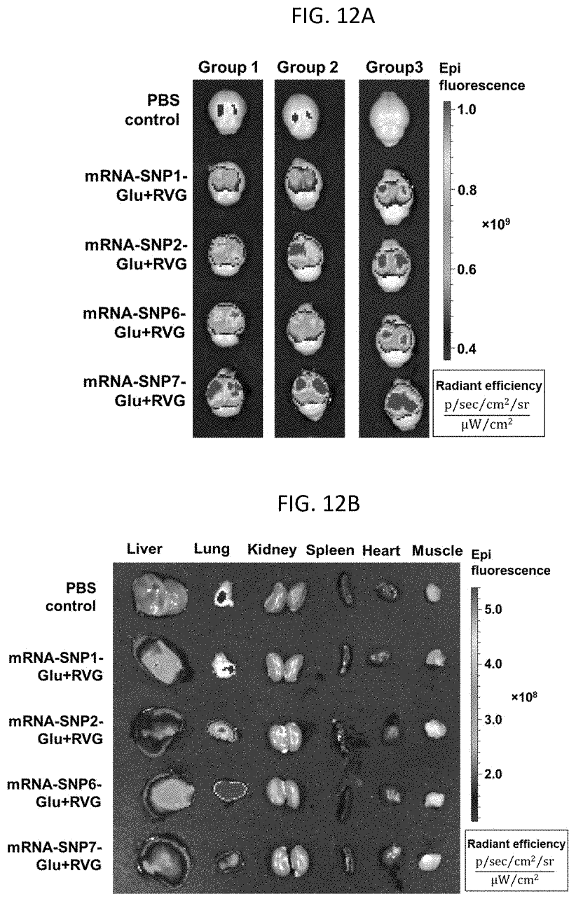

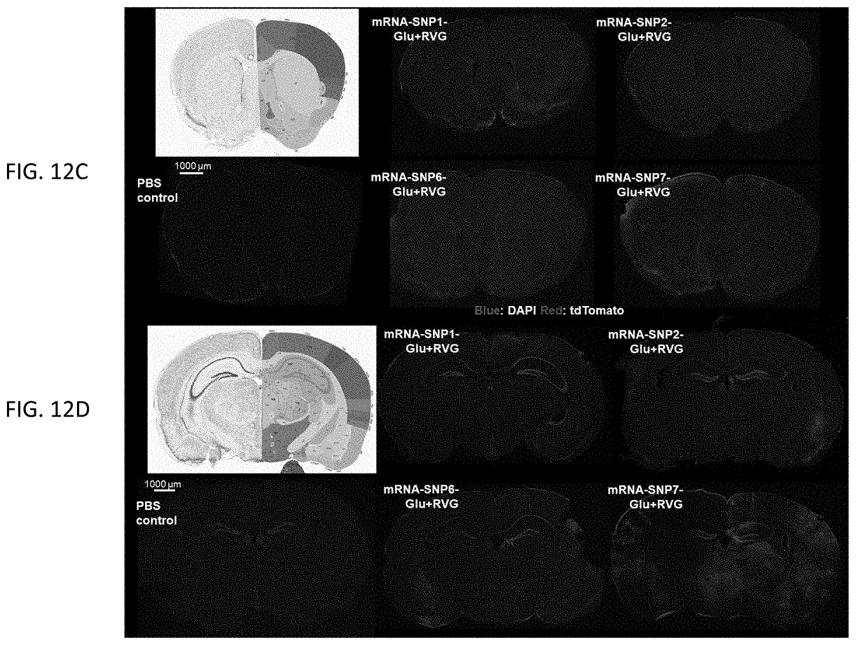

To further study the effects of the SNP formulations on the delivery efficiency of mRNA in vivo, SNPs with a higher mRNA delivery efficiency than Lipo 2000 (i.e., SNP2, SNP6 and SNP7), as well as SNP1 (as the control) were involved. While all four Cre mRNA-encapsulated SNP (i.e., mRNA-SNP) formulations showed tdTomato expression in the brain, mRNA-SNP7-Glu+RVG (i.e., with unsaturated inactive arm) exhibited the highest tdTomato fluorescence intensity (FIG. 12A). All the SNPs showed moderate Cre mRNA delivery in the liver, while SNPs with inactive arms showed tdTomato expression in the lung. Although mRNA-SNP6-Glu+RVG, showed the highest mRNA delivery efficiency in vitro, it only exhibited moderate mRNA delivery efficiency in the brain; instead, it had the tendency to accumulate in the lung (FIG. 12B), indicating that different SNPs may be targeted to different regions of the body.

The mRNA delivery efficiency of the SNPs in the brain was further analyzed by immunofluorescence staining. Brains were fixed, cryosectioned and immunofluorescence stained with cell markers (i.e., NeuN, GFAP and CD31), as well as anti-tdTomato antibody to confirm tdTomato expression. Coronal section mosaic tile image of the mouse brain had shown tdTomato expression in the whole brain (FIG. 12C). Notably, mRNA-SNP7-Glu+RVG induced wide spread and high-level tdTomato expression, especially in the cortex and hippocampus, suggesting the spread of SNPs in brain parenchyma after bypassing the BBB.

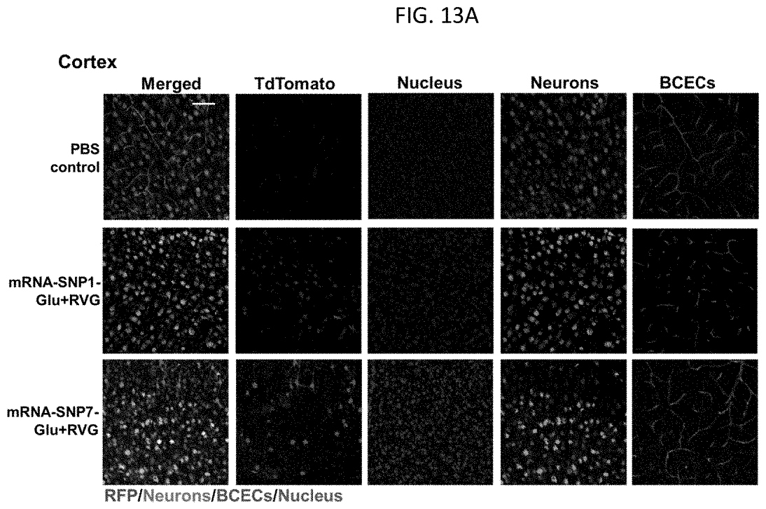

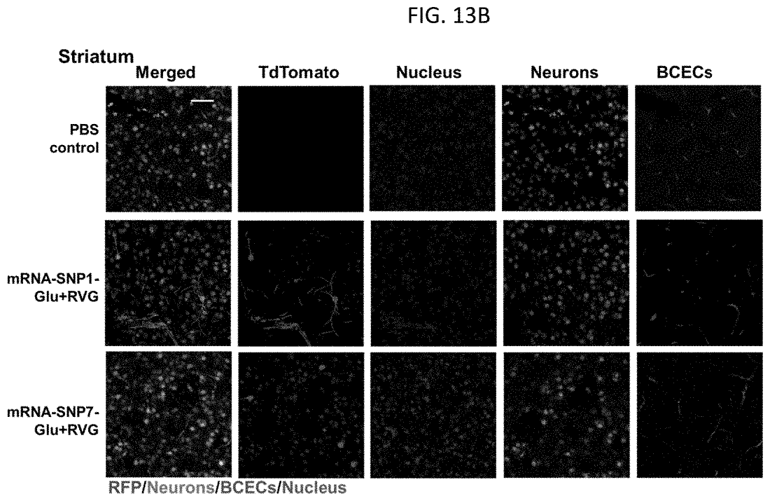

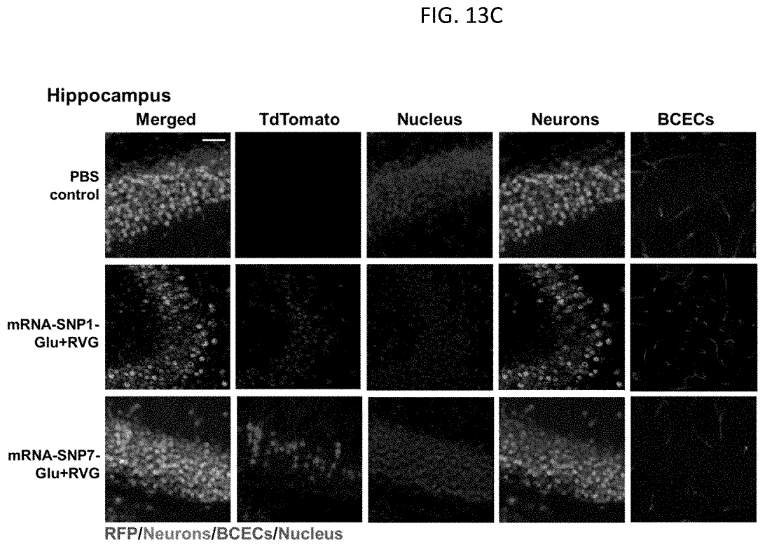

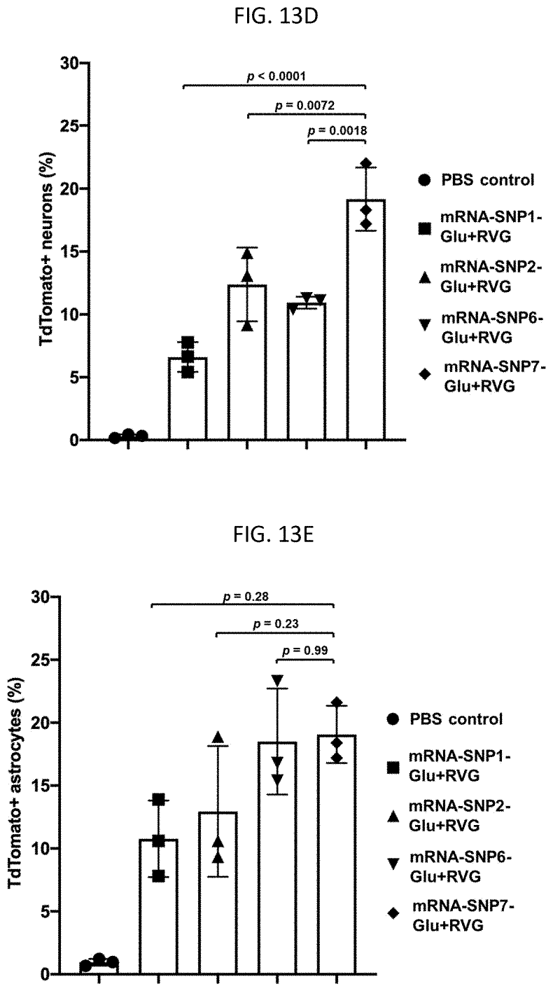

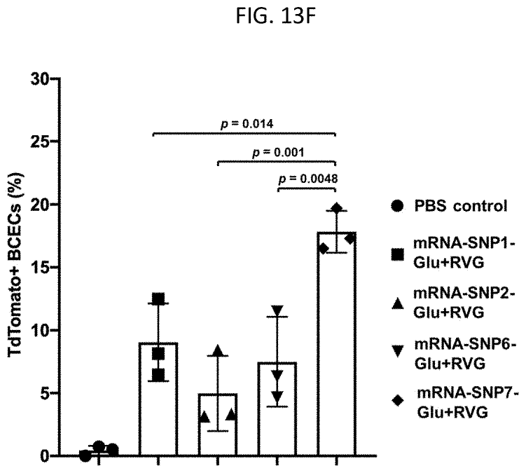

Triple-color immunofluorescence staining was used to identify the cell types that SNPs edited in vivo (FIG. 13). The SNP formulation with the highest tdTomato expression level was analyzed and compared to the PBS control and mRNA-SNP1-Glu+RVG. In the cortex of mRNA-SNP1-Glu+RVG treated brain, the majority of the tdTomato positive cells were overlapping with NeuN (neurons), while a small portion of the tdTomato positive cells were overlapping with CD31 (BCECs) (FIG. 13A). Other locations of the brain (i.e., striatum and hippocampus) also had similar results. In the mRNA-SNP7-Glu+RVG treated brains, the tdTomato expression level was significantly higher and there was a slight increase of tdTomato positive cells that were not co-localized with NeuN or CD31 in the cortex and striatum (FIGS. 13A and 13B), indicating mRNA-SNP7-Glu+RVG can also be internalized by other cell types, (e.g., astrocytes, microglial cells). However, in the hippocampus, mRNA-SNP7-Glu+RVG induced tdTomato expression were only found in neurons (FIG. 13C).

We next performed FACS to identify the cell types that are edited after SNP treatment and quantify the editing efficiency. SNP7 showed the highest neuron (21%) and BCEC (17%) editing efficiencies (FIGS. 13D, 13F), indicating it has the best capability to cross the BBB and target the brain among the four SNP formulations studied. Notably, SNP7 exhibited a slightly higher neuron editing efficiency than that of other cell types, likely attributed to the RVG neuronal targeting ligand. SNP6 exhibited higher editing efficiency in astrocytes in the brain in comparison with neurons and BECEs (FIG. 13E).

As shown by the examples above, SNPs of the present technology effectively deliver nucleic acids and genome editors. SNPs with different structures (i.e., SNPs made of different silica reagents) may be targeted to different regions of the body after systemic administration. With surface PEGylation and glucose/RVG conjugation, SNPs can bypass the BBB and induce payload delivery in different brain cells including neurons, astrocytes and BCECs, with up to 20% editing efficiency. Therefore, this technology may be used to treat many genetic neurological diseases via gene therapy or genome editing.

### EQUIVALENTS

While certain embodiments have been illustrated and described, a person with ordinary skill in the art, after reading the foregoing specification, can effect changes, substitutions of equivalents and other types of alterations to the nanoparticles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Likewise, the use of the terms “comprising,” “including,” “containing,” etc. shall be understood to disclose embodiments using the terms “consisting essentially of” and “consisting of.” The phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A nanoparticle comprising a silica network comprising crosslinked polysiloxanes, wherein:
the polysiloxanes comprise silyloxy subunits having the structure (I) 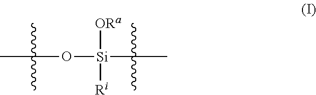
wherein
R^{a} at each occurrence is independently selected from a bond to a Si of another polysiloxane chain or a C₁₋₁₂ alkyl group;
Rⁱ at each occurrence is independently selected from the group consisting of C₁₋₁₂ alkyl and C₂₋₁₂ alkenyl groups, optionally substituted with a substituent selected from the group consisting of halogen and NR¹₂, wherein each occurrence of R¹ is independently selected from H or a C₁₋₁₂ alkyl group, or two R¹ groups, together with the N atom to which they are attached, form a pyrrolidine or piperidine ring;
the crosslinks between polysiloxanes comprise disulfide linkages,
the nanoparticle comprises an exterior surface comprising surface-modifying groups attached to and surrounding the silica network, wherein the surface-modifying groups comprise polyethylene glycol (PEG), polysarcosine, polyzwitterion, polycation, polyanion, or combinations of two or more thereof; and
the nanoparticle has an average diameter of 15 nm to 200 nm.

2. The nanoparticle of claim 1, wherein R^{a} at each occurrence is independently selected from a bond to a Si of another polysiloxane chain or a C₁₋₆ alkyl group.

3. The nanoparticle of claim 1, wherein Rⁱ at each occurrence is independently selected from the group consisting of C₁₋₁₂ alkyl and C₂₋₁₂ alkenyl groups, optionally substituted with a substituent selected from the group consisting of halo and NR¹₂, wherein each occurrence of R¹ is independently selected from H or a C₁₋₁₂ alkyl group.

4. The nanoparticle of claim 1, wherein Rⁱ is a C₁₋₄ alkyl group, optionally substituted with a halo or NR¹₂ substituent.

5. The nanoparticle of claim 1, wherein Rⁱ is a C₂₋₄ alkenyl group.

6. The nanoparticle of claim 1, wherein Rⁱ at each occurrence is independently selected from methyl, propyl, 3-chloropropyl, 3-aminopropyl, 3-dimethylaminopropyl, and vinyl.

7. The nanoparticle of claim 1, wherein the siloxy subunits of structure (I) are derived from one or more of triethoxymethylsilane, triethoxypropylsilane, triethoxy-3-chloropropylsilane, triethoxy-3-aminopropylsilane, triethoxy-3-dimethylaminopropylsilane, triethoxyoctylsilane, or triethoxyvinylsilane.

8. The nanoparticle of claim 1, wherein the polysiloxanes further comprise silyloxy subunits having structure (IVC): 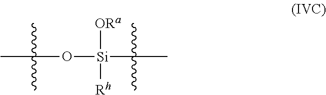
wherein
R^{a} at each occurrence is independently selected from a bond to a Si of another polysiloxane chain or a C₁₋₁₂ alkyl group; and
R^{h} at each occurrence is a moiety comprising a weakly basic group.

9. The nanoparticle of claim 8, wherein the weakly basic group is selected from imidazolyl, pyridinyl, tetrahydroquinolinyl, or indolinyl groups, or a combination any two or more thereof.

10. The nanoparticle of claim 8, wherein R^{h} has the structure —(CH₂)ₙ-L-Z, and wherein
L is a bond or is a linking group selected from —C(O)NH—, —O—, —NH—, —C(O)—, or —C(O)O;
Z is at each occurrence is independently a picolinyl, lutidinyl, indolinyl, tetrahydroquinolinyl, quinolinyl, imidazolyl, or pyridinyl group; and
n is 0, 1, 2, 3, or 4.

11. The nanoparticle of claim 8, wherein R^{h} has the structure (D):

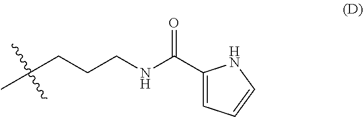 [SMILES: *CCCNC(=O)c1ccc[nH]1]

12. The nanoparticle of claim 1, wherein the crosslinked polysiloxanes comprise crosslinking subunits having the structure (V): 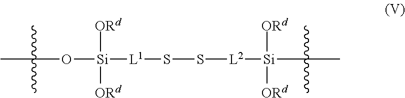
wherein:
L¹ and L² at each occurrence are independently selected from a C₁₋₆ alkylene group; and
R^{d} at each occurrence is independently selected from a bond to another polysiloxane chain or a C₁₋₆ alkyl group.

13. The nanoparticle of claim 12, wherein R^{d} at each occurrence is ethyl.

14. The nanoparticle of claim 12, wherein each of L¹ and L² is propylene at each occurrence.

15. The nanoparticle of claim 1, wherein the polysiloxanes comprise a plurality of siloxy subunits having the structure (VI): 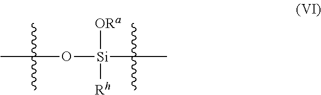
wherein:
R^{a} at each occurrence is selected from a bond to Si from another polysiloxane chain or a C₁₋₆ alkyl group, and
R^{e} at each occurrence is the surface-modifying group, optionally including a C₁₋₆ linker group connecting the surface-modifying group to the Si atom to which R^{e} is attached.

16. The nanoparticle of claim 15, wherein the C₁₋₆ linker group is present and connected to the surface-modifying group directly or via an amine, ether, amide, ester, urethane, urea, imine, or sulfide group.

17. The nanoparticle of claim 15, wherein the C₁₋₆ linker group is present and is —NHC(O)NH—(C₂₋₅ alkylene)-, —NHC(O)—(C₂₋₅ alkylene)-, —C(O)NH—(C₂₋₅ alkylene)-, —NH—(C₂₋₅ alkylene)-, —O—(C₂₋₅ alkylene)-, —S—(C₂₋₅ alkylene)-, —OC(O)NH—(C₂₋₅ alkylene)-, or —NHC(O)O—(C₂₋₅ alkylene)-.

18. The nanoparticle of claim 1, wherein the surface-modifying groups are PEG or polysarcosine.

19. The nanoparticle of claim 18, wherein the surface-modifying groups comprise PEG attached to a siloxy subunit having the structure (VII) 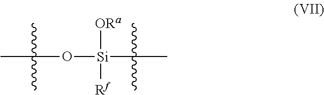
wherein R^{a} at each occurrence is selected from a bond to Si from another polysiloxane chain or a C₁₋₆ alkyl group, and R^{f} has the structure (E1): 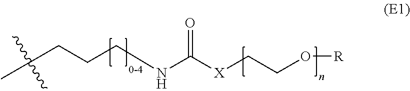
wherein X is O, NH, or CH₂O, and R is a C₁₋₆ alkyl, targeting ligand, a cell-penetrating peptide (CPP), or imaging agent.

20. The nanoparticle of claim 1, wherein the surface-modifying group is a polyzwitterion selected from poly(carboxybetaine methacrylate) (PCBMA)⋅poly(sulfobetaine methacrylate) (PSBMA), poly(2-methacryloyloxyethyl phosphorylcholine) (PMPC), or combinations of two or more thereof; or
the surface-modifying group is a cationic polymer selected from polyethyleneimine (PEI), polylysine, polyarginine, polyamidoamine (PAMAM), or combinations of two or more thereof; or
the surface-modifying group is an anionic polymer selected from poly(glutamic acid) or poly(acrylic acid).

21. The nanoparticle of claim 1, wherein the surface-modifying groups further comprise one or more of a targeting ligand, a CPP, or an imaging agent.

22. The nanoparticle of claim 1, wherein the surface-modifying groups further comprise a targeting ligand selected from a cofactor, carbohydrate, peptide, antibody, nanobody, or aptamer.

23. The nanoparticle of claim 22, wherein the targeting ligand is selected from the group consisting of glucose, RVG peptide, folic acid, mannose, GE11, cRGD, KE108, octreotide, PSMA aptamer, TRC105, 7D12 nanobody, all-trans retinoic acid (ATRA), 11-cis-retinal (11cRal), CTB, N-acetylgalactosamine (GalNAc) and combinations of two or more thereof.

24. The nanoparticle of claim 22, wherein the targeting ligand is selected from glucose, RVG peptide, or both.

25. The nanoparticle of claim 1, wherein the surface-modifying groups further comprise an imaging agent selected from the group consisting of fluorescent dyes, radioisotope chelators for PET imaging, chelators for MRI imaging.

26. The nanoparticle of claim 1, wherein the surface potential of the nanoparticle ranges from −45 mV to +45 mV.

27. The nanoparticle of claim 1, wherein the surface potential is −10 mV to +10 mV.

28. The nanoparticle of claim 1, wherein the average diameter is 20 nm to 70 nm.

29. The nanoparticle of claim 1, further comprising a water-soluble biomolecule non-covalently bound to the nanoparticle.

30. The nanoparticle of claim 29, wherein the water-soluble biomolecule is selected from the group consisting of a polynucleic acid, polypeptide, a polynucleic acid/polypeptide complex and combinations of two or more thereof.

31. The nanoparticle of claim 29, wherein the water-soluble biomolecule is selected from the group consisting of DNA, RNA, and a ribonucleoprotein complex (RNP).

32. The nanoparticle of claim 31, wherein the water-soluble biomolecule is selected from RNP, plasmid DNA (pDNA), single-stranded donor oligonucleotide (ssODN), complementary (cDNA), messenger RNA (mRNA), small interfering RNA (siRNA), microRNA (miRNA), short hairpin RNA (shRNA), single guide RNA (sgRNA), transfer RNA (tRNA), ribozymes, and combinations of two or more thereof.

33. The nanoparticle of claim 31, wherein the water-soluble biomolecule is Cas9 RNP, Cas9 RNP+ssODN or a base editor.

34. The nanoparticle of claim 30, wherein the water-soluble biomolecule is a polypeptide.

35. A method of delivering a water-soluble biomolecule into a cell comprising exposing the cell to a nanoparticle of claim 1.

36. A method of treating a condition or disorder in a subject that may be ameliorated by a biomolecule comprising administering to the subject an effective amount of a nanoparticle of claim 1.

37. The method of claim 36, wherein the condition or disorder occurs in the central nervous system of the subject, and the nanoparticle comprises glucose and/or RVG peptide targeting ligands.
